SEQUENCE LISTING (4 sequences)

SEQ ID NO: 1 - RNA, 27 nt, Artificial Sequence
  Synthetic Polynucleotide
uacacucgaucuagacuaaagcugcuc

SEQ ID NO: 2 - RNA, 27 nt, Artificial Sequence
  Synthetic Polynucleotide
uacacuccaucuaaacuaaaccuccuc

SEQ ID NO: 3 - RNA, 27 nt, Artificial Sequence
  Synthetic Polynucleotide
gagcagcuuuaguuuagaucgagugua

SEQ ID NO: 4 - RNA, 12 nt, Artificial Sequence
  Synthetic Polynucleotide
cuguggccugcu

(19) United States
(12) Patent Application Publication
(10) Pub. No.: US 2022/0143198 A1
(43) Pub. Date: May 12, 2022

(54) COMPOSITIONS AND METHODS RELATED TO TETHERED KETHOXAL DERIVATIVES

(71) Applicant: The University of Chicago, Chicago, IL (US)

(72) Inventors: Chuan HE, Chicago, IL (US); Tong WU, Chicago, IL (US); Pingluan WANG, Chicago, IL (US)

(73) Assignee: The University of Chicago, Chicago, IL (US)

(21) Appl. No.: 17/595,477

(22) PCT Filed: May 22, 2020
(86) PCT No.: PCT/US2020/070073

§ 371 Date: Nov. 17, 2021

Related U.S. Application Data
(60) Provisional application No. 62/987,932, filed on Mar. 11, 2020.
(60) Provisional application No. 62/851,386, filed on May 22, 2019.

(51) Int. Cl. A61K 47/54; A61K 31/121; A61K 47/55

(52) U.S. Cl. CPC A61K 47/557; A61K 47/549; A61K 47/55; A61K 31/121

(57) ABSTRACT

Embodiments are directed to therapeutic, diagnostic, or functional complexes comprising a kethoxal derivative.

### CROSS-REFERENCE TO RELATED APPLICATIONS

This application claims the benefit of priority of U.S. Provisional Patent Application No. 62/851,386 filed May 22, 2019, and U.S. Provisional Patent Application No. 62/987,932 filed Mar. 11, 2020, all of which are hereby incorporated by reference in their entirety.

### STATEMENT REGARDING FEDERALLY FUNDED RESEARCH

This invention was made with government support under HG008935 awarded by the National Institutes of Health. The government has certain rights in the invention.

### BACKGROUND OF THE INVENTION

### Field of the Invention

Embodiments generally concern molecular and cellular biology. In particular, embodiments are directed to methods and composition for labeling nucleic acids.

### SUMMARY OF THE INVENTION

Click chemistry kethoxal derivatives (“kethoxal derivatives”)(e.g., N₃-kethoxal) have been developed that efficiently couple to single-stranded DNAs and/or RNAs in live cells by reacting with the Watson-Crick interface of guanine bases. The labelling product can be further functionalized and enriched, for example using biotin/biotin binding partner or other agents.

Certain embodiments are directed to a complex(es) of an agent or binding moiety (e.g., a therapeutic (small molecule, nucleic acid, peptide, etc.), diagnostic (imaging agent, etc.), or functional agent (probe, label etc.)) coupled to a kethoxal derivative. In certain aspects, a compound/kethoxal derivative can have the following general formula:

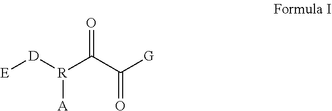

In certain aspects, a compound/kethoxal derivative can have the general formula of Formula I, wherein E is selected from a reactive group, click chemistry moiety, binding group, or therapeutic agent; D is optionally a linker or a direct bond; R is a connecting element or group; A is a substituent or a second E moiety selected independent of the first E moiety; and G is a dicarbonyl-defining group.

In certain aspects, R can be selected from substituted or unsubstituted carbon, nitrogen, aryl, alkylaryl, or heterocyclic group.

In certain aspects, A can be substituted with one or more (mono-substituted, di-substituted, etc.) of H, F, CF₃, CF₂H, CFH₂, CH₃, alkyl group, or combinations thereof. In certain aspects, A can be mono- or di-substituted with a linker. In certain aspects, A can be mono- or di-substituted with a reactive group, e.g., a click chemistry moiety, therapeutic agent, or binding moiety. In other aspects, A can be a second E group (E₂ relative to an E₂).

In certain aspects, D is a linker selected from an ester, amide, tetrazine, tetrazole, triazine, triazole, aryl groups, heterocycle, sulfonamide, thiourea, a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. D can be —N(CH₃)—, —OCH₂—, —N(CH₃)COCH₂—, or a group having the chemical formula of Formula VII. In certain instances, the linker can be a concatamer (comprising 1, 2, 3, 4, 5, 6, 7, 8, 9, 10 or more linker(s)) of 1, 2, 3, 4, 5, 6, 7, 8, 9, 10, or more of the linkers described above.

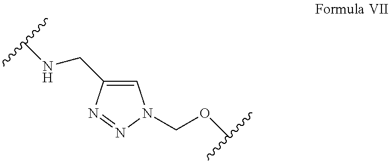 [SMILES: *NCc1cn(CO*)nn1]

In some aspects, D can be substituted with a reactive group, e.g., a click chemistry moiety. In some aspects, In some aspects, D can be a direct bond between E and R. In certain aspects, D can be a substituent that modulates the stability of the product formed, including alkoxy groups, ethers, carbonyls, aryl groups, electron withdrawing or electron donating groups, electrophilic of nucleophilic centers, or H-bond acceptors.

In certain aspects, G can be independently selected from H, F, CF₃, CF₂H, CFH₂, CH₃, or alkyl group.

In certain aspects, E can be selected from alkynes, azides, strained alkynes, dienes, dieneophiles, alkoxyamines, carbonyls, phosphines, hydrazides, thiols, alkenes, diazirines. In some aspects, E can be a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain aspects, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazrines, dienes, dieneophiles, alkoxyamines, carbonyls, phosphines, hydrazides, thiols, and alkenes. In certain aspects, E can be further coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

Specific compounds include, but are not limited to a compound of Formula I where (i) G is H, R is C, A is methyl, D is —OCH₂CH₂-triazole-pyridine-aryl-amide-CH₂CH₂, and E is N₃ (azide); (ii) G is H; R is C, A is F, D is —OCH₂CH₂-triazole-amide-benzoimidazole-phenyl-NHCO—CH₂CH₂, and E is alkyne; (iii) G is H, R is C, A is a di-fluoro substituent of R, D is —OCH₂CH₂-triazole-CH₂-pyridine-benzoimidazole-NHCO—CH₂CH₂CH₂—, and E is N₃ (azide); (iv) G is H, R is C, A is methyl, D is —OCH₂CH₂-triazole-, and E is phenol or diphenol.

In certain aspects, the kethoxal complex is selected from 3-azido-2-oxopropanal, 3-azido-2-oxobutanal, 3-azido-3-fluoro-2-oxopropanal, 2-oxo-6-(2-oxohexahydro-1H-thieno[3,4-d]imidazol-4-yl)hexanal, 2-((1S,4S)-bicyclo[2.2.1]hept-5-en-2-yl)-2-oxoacetaldehyde, 2-oxo-2-phenylacetaldehyde, 2-(3,5-dimethoxyphenyl)-2-oxoacetaldehyde, 2-(4-nitrophenyl)-2-oxoacetaldehyde, N-(2,3-dioxopropyl)-N-methyl-5-(2-oxohexahydro-1H-thieno[3,4-d]imidazol-4-yl)pentanamide, N-((1-(2-((3,4-dioxobutan-2-yl)oxy)ethyl)-1H-1,2,3-triazol-4-yl)methyl)-5-(2-oxohexahydro-1H-thieno[3,4-d]imidazol-4-yl)pentanamide, 2-oxo-3-(prop-2-yn-1-yloxy)butanal, (E)-3-(2-(cyclooct-4-en-1-ylamino)ethoxy)-2-oxobutanal, 3-(2-azidoethoxy)-2-oxopropanal, 3,4-dioxobutan-2-yl 2-azidoacetate, 3-(2-azidoethoxy)-3-methyl-2-oxobutanal, 5-azido-2-oxopentanal, 2-azido-N-(3,4-dioxobutan-2-yl)-N-methylacetamide, 3-(2-azidoethoxy)-2-oxobutanal, 3-(2-azidoethoxy)-3-fluoro-2-oxopropanal, 3-(2-azidoethoxy)-3,3-difluoro-2-oxopropanal, 4-(2-azidoethoxy)-2-oxobutanal, or 3-(((1S,4S)-bicyclo[2.2.1]hept-5-en-2-yl)methoxy)-2-oxobutanal. Any 1, 2, 3, 4, 5, 6, 7, 8, 9, or 10 of these compounds can be explicitly excluded.

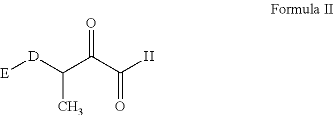

In certain aspects, a compound/kethoxal derivative can have the general formula of Formula II, wherein E is selected from a reactive group, click chemistry, binding group, or therapeutic agent; and D is optionally a linker or a direct bond.

In certain aspects, D is a linker selected from an ester, amide, tetrazine, tetrazole, triazine, triazole, aryl groups, heterocycle, sulfonamide, a substituted or unsubstituted —(CH₂)ₙ— where n is 1-10 with 0, 1, 2, 3, 4, 5, 6, 7, 8, 9, 10 methyl substitutions; —O(CH₂), where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. In some aspects, D can be —N(CH₃)—, —OCH₂—, —N(CH₃)COCH₂—, or a group having the chemical formula of Formula VII. In certain instances, the linker can be a concatamer (comprising 1, 2, 3, 4, 5, 6, 7, 8, 9, 10 or more linker(s)) of 1, 2, 3, 4, 5, 6, 7, 8, 9, 10, or more of the linkers described above.

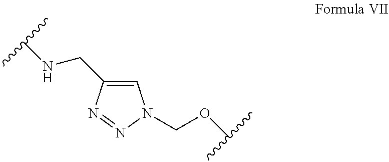 [SMILES: *NCc1cn(CO*)nn1]

In some aspects, D can be substituted with a reactive group, e.g., a click chemistry moiety. In some aspects, D can be a direct bond between E and the carbon atom binding A. In certain aspects, D can be a substituent that modulates the stability of the product formed, selected from alkoxy groups, ethers, carbonyls, aryl groups, electron withdrawing groups (e.g., nitro-, trifluoromethyl-, cyano groups, trimethylsilyl-, esters—either as stand-alone substituents or substituted aryl groups) or electron donating groups (e.g., alkyl groups, thiols, amines, aziridines, oxiranes, alkenes—either as stand-alone substituents or substituted aryl groups), electrophilic or nucleophilic centers (e.g., aldehydes, ketones, anhydrides, imines, nitriles, alkenes, alkynes, aryls, heteroaryls), or H-bond acceptors or donors (e.g., ethers, alcohols, carbonyls, amines, thiols, thioethers, sulfonamides, halides).

In certain aspects, E is selected from a reactive group, click chemistry, binding group, or therapeutic agent. In certain instances, E can be selected from alkynes, azides, strained alkynes, dienes, dieneophiles, alkoxyamines, carbonyls, phosphines, hydrazides, thiols, alkenes, diazirines. In some aspects, E can be a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain aspects, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazrines, dienes, dieneophiles, alkoxyamines, carbonyls, phosphines, hydrazides, thiols, and alkenes. In certain aspects, E can be further coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

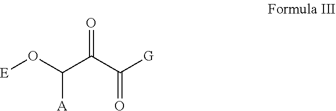

In certain aspects, a compound/kethoxal derivative can have the general formula of Formula III, where E is selected from a reactive group, click chemistry moiety, binding group, or therapeutic agent; A is a substituent or a second E moiety selected independent of the first E moiety; and G is a dicarbonyl-defining group.

In certain aspects, E is a click chemistry moiety selected from alkynes, azides, strained alkynes, dienes, dieneophiles, alkoxyamines, carbonyls, phosphines, hydrazides, thiols, alkenes, and diazirines. In certain aspects, E can be selected from alkynes, azides, strained alkynes, dienes, dieneophiles, alkoxyamines, carbonyls, phosphines, hydrazides, thiols, alkenes, diazirines. In some aspects, E can be a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain aspects, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazrines, dienes, dieneophiles, alkoxyamines, carbonyls, phosphines, hydrazides, thiols, and alkenes. In some aspects, E can further comprise a linker (E can be a reactive group having a terminal click chemistry moiety).

In certain aspects, A can be a linker (as defined for D), A can be further coupled to an agent or binding moiety. A or G can be independently selected from H, F, CF₃, CF₂H, CFH₂, CH₃, or alkyl group. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

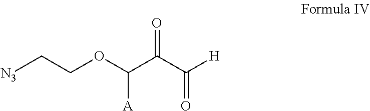

In certain aspects, a compound/kethoxal derivative can have the general formula of Formula IV, wherein A is a substituent or a second E moiety selected independent of the first E moiety. In certain aspects, A is substituted with one or more (mono-substituted, di-substituted, etc.) of H, F, CF₃, CF₂H, CFH₂, CH₃, alkyl group, or combinations thereof. In certain aspects, A can be mono- or di-substituted with a linker. In certain aspects, A can be mono- or di-substituted with a reactive group, e.g., a click chemistry moiety, therapeutic agent, or binding moiety. In certain aspects, the azide moiety is further coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

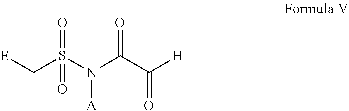

In certain aspects, a compound/kethoxal derivative can have the general formula of Formula V, wherein E is selected from a reactive group, click chemistry moiety, binding group, or therapeutic agent, and A is a substituent or a second E moiety selected independent of the first E moiety.

In certain aspects, E is a click chemistry moiety selected from alkynes, azides, strained alkynes, dienes, dieneophiles, alkoxyamines, carbonyls, phosphines, hydrazides, thiols, alkenes, and diazirines. In certain aspects, E can be selected from alkynes, azides, strained alkynes, dienes, dieneophiles, alkoxyamines, carbonyls, phosphines, hydrazides, thiols, alkenes, diazirines. In some aspects, E can be a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain aspects, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazrines, dienes, dieneophiles, alkoxyamines, carbonyls, phosphines, hydrazides, thiols, and alkenes. In certain aspects, E can be further coupled to a linker (E can be a linker having a terminal click chemistry moiety).

A is substituted with one or more (mono-substituted, di-substituted, etc.) of H, F, CF₃, CF₂H, CFH₂, CH₃, alkyl group, or combinations thereof. In certain aspects, A can be mono- or di-substituted with a linker. In certain aspects, A can be mono- or di-substituted with a reactive group, e.g., a click chemistry moiety, therapeutic agent, or binding moiety. In certain aspects, the azide moiety is further coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

In certain aspects E, A, or E and A can be independently coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

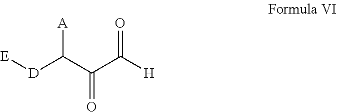

In certain aspects, a compound/kethoxal derivative can have the general formula of Formula VI, wherein A can be substituted with one or more or H, F, CF₃, CF₂H, CFH₂, CH₃, alkyl group or combinations thereof; D is optionally a linker or a direct bond; and E can be a be a reactive functional group. In certain aspects, A is a substituent or a second E moiety selected independent of the first E moiety.

In certain aspects, E is a click chemistry moiety selected from alkynes, azides, strained alkynes, dienes, dieneophiles, alkoxyamines, carbonyls, phosphines, hydrazides, thiols, alkenes, and diazirines. In certain aspects, E can be selected from alkynes, azides, strained alkynes, dienes, dieneophiles, alkoxyamines, carbonyls, phosphines, hydrazides, thiols, alkenes, diazirines. E can be a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain aspects, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazrines, dienes, dieneophiles, alkoxyamines, carbonyls, phosphines, hydrazides, thiols, and alkenes. In certain aspects, E can be further coupled to a linker (E can be a linker having a terminal click chemistry moiety).

In certain aspects, D is a linker selected from an ester, amide, tetrazine, tetrazole, triazine, triazole, aryl groups, heterocycle, sulfonamide, a substituted or unsubstituted —(CH₂)ₙ— where n is 1-10 with 0, 1, 2, 3, 4, 5, 6, 7, 8, 9, 10 methyl substitutions; —O(CH₂), where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. In some aspects, D can be —N(CH₃)—, —OCH₂—, —N(CH₃)COCH₂—, or a group having the chemical formula of Formula VII. In certain instances, the linker can be a concatamer (comprising 1, 2, 3, 4, 5, 6, 7, 8, 9, 10 or more linker(s)) of 1, 2, 3, 4, 5, 6, 7, 8, 9, 10, or more of the linkers described above.

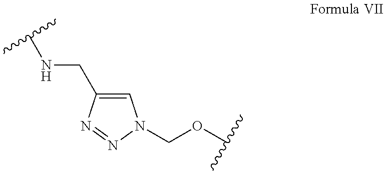 [SMILES: *NCc1cn(CO*)nn1]

In some aspects, D can be substituted with a reactive group, e.g., a click chemistry moiety. In some aspects, D can be a direct bond between E and the carbon atom binding A. In certain aspects, D can be a substituent that modulates the stability of the product formed, selected from alkoxy groups, ethers, carbonyls, aryl groups, electron withdrawing groups (e.g., nitro-, trifluoromethyl-, cyano groups, trimethylsilyl-, esters—either as stand-alone substituents or substituents on aryl groups) or electron donating groups (e.g., alkyl groups, thiols, amines, aziridines, oxiranes, alkenes—either as stand-alone substituents or substituents on aryl groups), electrophilic or nucleophilic centers (e.g., aldehydes, ketones, anhydrides, imines, nitriles, alkenes, alkynes, aryls, heteroaryls), or H-bond acceptors or donors (e.g., ethers, alcohols, carbonyls, amines, thiols, thioethers, sulfonamides, halides).

A is substituted with one or more (mono-substituted, di-substituted, etc.) of H, F, CF₃, CF₂H, CFH₂, CH₃, alkyl group, or combinations thereof. In certain aspects, A can be mono- or di-substituted with a linker. In certain aspects, A can be mono- or di-substituted with a reactive group, e.g., a click chemistry moiety, therapeutic agent, or binding moiety. In certain aspects, the azide moiety is further coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

In all the formulations provided herein, reactive groups can be activated by pH changes, oxidation, light, metal or other catalysts. In certain aspects E can contain a detectable label including, but not limited to: a drug, a toxin, a peptide, a polypeptide, an epitope tag, a member of a specific binding pair, a fluorophore, a solid support, a nucleic acid (DNA/RNA), a lipid, or a carbohydrate. In certain aspects, E can contain an affinity group including biotin (or the tetrahydro-1H-thieno[3,4-d]imidazol-2(3H)-one moiety on biotin), ligand, substrate, macromolecule with affinity to another molecule, macromolecule, or surface. In certain aspects, E can be a group having the chemical formula of Formula VIIIA-F, shown in FIG. 2A FIG. 2B provides examples of such compounds of Formula VI.

The complex can tether an agent or binding moiety to a nucleic, and as such the kethoxal derivative acts a tether between a functional agent and a nucleic in proximity to the functional agent. The kethoxal derivative is a tether or bifunctional entity, which can be called a biofunctional moiety. The agent can be a small molecule, oligonucleotide, or the like. In certain aspects the agent, binding moiety, or small molecule binds to a protein or a nucleic acid. In certain aspects, the agent is a therapeutic agent. The therapeutic agent can be a small molecule, drug, medicine, pharmaceutical, hormone, antibiotic, protein, gene, nucleic acid growth factor, bioactive material, etc., used for treating, controlling, or preventing diseases or medical conditions. In other aspects, the agent or therapeutic agent is a nucleic acid. The nucleic acid can be an inhibitory nucleic acid, for example a siRNA. The kethoxal derivative can be a N₃-kethoxal and can be operatively couple to agent or binding agent.

Certain embodiments are directed to methods for localizing an agent or therapeutic agent to a nucleic acid comprising contacting a cell with a complex or biofunctional complex described herein.

The kethoxal derivatives and their complexes can be used in vivo, ex vivo or in vitro. As used herein the term “in vivo” refers to any process/event that occurs within a living subject. As used herein the term “in vitro” refers to any process/event that occurs outside a living subject in an artificial environment, e.g., without limitation, in a test tube or culture medium. In some embodiment, in vitro refers to cell lines grown in cell culture. In some embodiment, in vitro refers to tumor cells grown in cell culture. In some embodiments in vitro refers to components in an assay or composition that is not associated with a living cell. The term “ex vivo” refers to a cell or tissue culture technique using biological samples taken from a body.

Certain embodiments are directed to methods for localizing an agent or therapeutic agent in a cell including (i) contacting a target cell with a complex or biofunctional complex described herein to form a treated cell; (ii) coupling the complex or biofunctional complex to a nucleic acid through a kethoxal derivative that couples to guanine base(s).

The term “kethoxal derivative” refers to a compound having the basic backbone structure of kethoxal [—(O)C—C(O)—] with additional substituents added to that backbone structure.

The term “nucleoside” and “nucleotide” refers to a compound having a pyrimidine nucleobase, for example cytosine (C), uracil (U), thymine (T), inosine (I), or a purine nucleobase, for example adenine (A) or guanine (G), linked to the C-1′ carbon of a “natural sugar” (i.e., -ribose, 2′-deoxyribose, and the like) or sugar analogs thereof, including 2′-deoxy and 2′-hydroxyl forms. Typically, when the nucleobase is C, U or T, the pentose sugar is attached to the N1-position of the nucleobase. When the nucleobase is A or G, the ribose sugar is attached to the N9-position of the nucleobase (Kornberg and Baker, DNA Replication, 2nd Ed., Freeman, San Francisco, Calif., (1992)). The term “nucleotide” as used herein refers to a phosphate ester of a nucleoside as a monomer unit or within a polynucleotide, e.g., triphosphate esters, wherein the most common site of esterification is the hydroxyl group attached at the C-5′ position of the ribose.

As used herein the term “agent” include chemical moieties that are coupled to a kethoxal derivate and include therapeutic agents, diagnostic agents and/or functional agents.

As used herein, a “therapeutic agent” is a molecule or atom which is conjugated to a kethoxal derivative to produce a conjugate or complex that is useful for therapy. Non-limiting examples of therapeutic agents include drugs, prodrugs, toxins, enzymes, enzymes that activate prodrugs to drugs, enzyme-inhibitors, nucleases, hormones, hormone antagonists, immunomodulators, e.g., cytokines, i.e., interleukins, such as interleukin-2, lymphokines, interferons and tumor necrosis factor, oligonucleotides (e.g., antisense oligonucleotides or interference RNAs, i.e., small interfering RNA (siRNA)), chelators, boron compounds, photoactive agents or dyes, radioisotopes or radionuclides.

Suitable additionally administered drugs, prodrugs, and/or toxins may include aplidin, azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin and analogs thereof, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, semustine streptozocin, tamoxifen, taxanes, taxol, testosterone propionate, thalidomide, thioguanine, thiotepa, teniposide, topotecan, uracil mustard, vinblastine, vinorelbine, vincristine, ricin, abrin, ribonuclease, ribonuclease, such as onconase, rapLR1, DNase I, Staphylococcal enterotoxin-A, pokeweed antiviral protein, gelonin, diphtheria toxin, Pseudomonas exotoxin, Pseudomonas endotoxin, nitrogen mustards, ethyleneimine derivatives, alkyl sulfonates, nitrosoureas, triazenes, folic acid analogs, anthracyclines, COX-2 inhibitors, pyrimidine analogs, purine analogs, antibiotics, epipodophyllotoxins, platinum coordination complexes, vinca alkaloids, substituted ureas, methyl hydrazine derivatives, adrenocortical suppressants, antagonists, endostatin or combinations thereof.

Suitable radionuclides may include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Se, ⁷⁷As, ⁸⁶Y, ⁸⁹Sr, ⁸⁹Zr, ⁹⁰Y, ⁹⁴Tc, ^{94m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹Pb ²¹²Bi, ²¹²Pb, ²¹³Bi, ²²³Ra, ²²⁵Ac, or mixtures thereof. If the radionuclide is to be used therapeutically, it may be desirable that the radionuclide emit 70 to 700 keV gamma particles or positrons. If the radionuclide is to be used diagnostically, it may be desirable that the radionuclide emit 25-4000 keV gamma particles and/or positrons. The radionuclide may be used to perform positron-emission tomography (PET), and the method may include performing PET.

Suitable photoactive agents and dyes, include agents for photodynamic therapy, such as a photosensitizer, such as benzoporphyrin monoacid ring A (BPD-MA), tin etiopurpurin (SnET2), sulfonated aluminum phthalocyanine (AISPc) and lutetium texaphyrin (Lutex).

As used herein, a “diagnostic agent” is a molecule or atom which is conjugated to a kethoxal derivative that is useful for diagnosis or imaging. Non-limiting examples of diagnostic agents include a photoactive agent or dye, a radionuclide, a radioopaque material, a contrast agent, a fluorescent compound, an enhancing agent (e.g., paramagnetic ions) for magnetic resonance imaging (MM) and combinations thereof. Suitable enhancing agents are Mn, Fe and Gd.

The therapeutic and/or diagnostic agent may be directly associated with the kethoxal derivative (e.g., covalently or non-covalently bound thereto).

“Nucleoside analog” and “nucleotide analog” refer to compounds having modified nucleobase moieties (e.g., pyrimidine nucleobase analogs and purine nucleobase analogs described below), modified sugar moieties, and/or modified phosphate ester moieties (e.g., see Scheit, Nucleoside Analogs, John Wiley and Sons, (1980); F. Eckstein, Ed., Oligonucleotides and Analogs, Chapters 8 and 9, IRL Press, (1991)). The ribose or ribose analog may be substituted or unsubstituted. Substituted ribose sugars include, but are not limited to, those riboses in which one or more of the carbon atoms, such as the 2′-carbon atom or the 3′-carbon atom, can be substituted with one or more of the same or different substituents such as —R, —OR, —NRR or halogen (e.g., fluoro, chloro, bromo, or iodo), where each R group can be independently —H, C1-C6 alkyl or C3-C14 aryl. Particularly, riboses are ribose, 2′-deoxyribose, 2′,3′-dideoxyribose, 3′-haloribose (such as 3′-fluororibose or 3′-chlororibose) and 3′-alkylribose, arabinose, 2′-O-methyl ribose, and locked nucleoside analogs (see for example PCT publication WO 99/14226), although many other analogs are also known in the art.

The term “nucleic acid” as used herein can refer to the nucleic acid material itself and is not restricted to sequence information (i.e., the succession of letters chosen among the five base letters A, C, G, T, or U) that biochemically characterizes a specific nucleic acid, for example, a DNA or RNA molecule. Nucleic acids described herein are presented in a 5′→3′ orientation unless otherwise indicated.

As used herein, the term “polynucleotide” refers to polymers of natural nucleotide monomers or analogs thereof, including double and single stranded deoxyribonucleotides, ribonucleotides, α-anomeric forms thereof, and the like. The terms “polynucleotide”, “oligonucleotide” and “nucleic acid” are used interchangeably. Usually the nucleoside monomers are linked by internucleotide phosphodiester linkages, whereas used herein, the term “phosphodiester linkage” refers to phosphodiester bonds or bonds including phosphate analogs thereof, and include associated counter-ions, including but not limited to H+, NH₄+, NR₄+, Na+, if such counter-ions are present. A polynucleotide may be composed entirely of deoxyribonucleotides, entirely of ribonucleotides or a mixture thereof.

“RNA” refers to ribonucleic acid and is a polymeric molecule implicated in various biological roles in coding, decoding, regulation, and expression of genes. RNA plays an active role within cells by catalyzing biological reactions, controlling gene expression, or sensing and communicating responses to cellular signals. Messenger RNA carries the information for the amino acid sequence of a protein to a ribosome, through which it is translated that the protein synthesized.

“DNA” refers to deoxyribonucleic acid and is a polymeric molecule present in nearly all living organisms as the main constituent of chromosomes as the carrier of genetic information. In various embodiments, the term DNA refers to genomic DNA, recombinant DNA, synthetic DNA, or complementary DNA (cDNA). In one embodiment, DNA refers to genomic DNA or cDNA. In particular embodiments, the DNA is a DNA fragment.

The term “click chemistry” refers to a chemical philosophy introduced by K. Barry Sharpless, describing chemistry tailored to generate covalent bonds quickly and reliably by joining small units comprising reactive groups together. Click chemistry does not refer to a specific reaction, but to a concept including reactions that mimic reactions found in nature. In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. A distinct exothermic reaction makes a reactant “spring loaded”. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation).

The term “click chemistry handle” or “click chemistry moiety”, as used herein, refers to a reactant, or a reactive group, that can partake in a click chemistry reaction. For example, an azide is a click chemistry handle. In general, click chemistry reactions require at least two molecules comprising complementary click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein. Other suitable click chemistry handles are known to those of skill in the art.

The term “linker,” as used herein, refers to a chemical group or molecule covalently linked to another molecule. In some embodiments, the linker is positioned between, or flanked by, two groups, molecules, or moieties and connected to each one via a covalent bond, thus connecting the two. In some embodiments, the linker is an organic molecule, group, or chemical moiety.

The term “stabilizing substituent” refers to a substituent that stabilizes/destabilizes a product (after reacting kethoxal derivatives with targets) through steric or electronic effects, such as hydrogen bonding, addition of electron-withdrawing or electron-donating groups, Michael acceptors, etc.

As used herein, the term “tag” or “affinity tag” refers to a moiety that can be attached to a compound, nucleotide, or nucleotide analog, and that is specifically bound by a partner moiety. The interaction of the affinity tag and its partner provides for the detection, isolation, etc. of molecules bearing the affinity tag. Examples include, but are not limited to biotin or iminobiotin and avidin or streptavidin. A sub-class of affinity tag is the “epitope tag,” which refers to a tag that is recognized and specifically bound by an antibody or an antigen-binding fragment thereof. Examples of suitable tags include, but are not limited to, amino acids, peptides, proteins, nucleic acids, polynucleotides, sugars, carbohydrates, polymers, lipids, fatty acids, and small molecules. Other suitable tags will be apparent to those of skill in the art and the invention is not limited in this aspect. In some embodiments, a tag comprises a sequence useful for purifying, expressing, solubilizing, and/or detecting a target. In some embodiments, a tag can serve multiple functions. In some embodiments, a tag comprises an HA, TAP, Myc, 6×His, Flag, or GST tag, to name few examples. In some embodiments, a tag is cleavable, so that it can be removed. In some embodiments, this is achieved by including a protease cleavage site in the tag, e.g., adjacent or linked to a functional portion of the tag. Exemplary proteases include, e.g., thrombin, TEV protease, Factor Xa, PreScission protease, etc. In some embodiments, a “self-cleaving” tag is used.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” is defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The term “effective,” as that term is used in the specification and/or claims, means adequate to accomplish a desired, expected, or intended result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of material is 10 mol. % of component.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Any embodiment disclosed herein can be implemented or combined with any other embodiment disclosed herein, including aspects of embodiments for compounds can be combined and/or substituted and any and all compounds can be implemented in the context of any method described herein. Similarly, aspects of any method embodiment can be combined and/or substituted with any other method embodiment disclosed herein. Moreover, any method disclosed herein may be recited in the form of “use of a composition” for achieving the method. It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-F: N₃-kethoxal and experimental evaluation of its selectivity, cell permeability and reversibility. (a) The structure of N₃-kethoxal and the reaction with guanine. (b) Denaturing gel electrophoresis demonstrating N₃-kethoxal only react with single-strand RNA (ssRNA). (c) Mass spectrum analysis of RNA oligos react with N₃-kethoxal. In RNA 1 with four guanines, all guanines and only guanine were labelled by N₃-kethoxal. In RNA 2 without guanine, no N₃-kethoxal labelling was observed. (d) Upper: Denaturing gel electrophoresis analysis of the labelling reaction of kethoxal and N₃-kethoxal with FAM-RNA oligo (5′-FAM-GAGCAGCUUUAGUUUAGAUCGAGUGUA (SEQ ID NO:3, lane 1-3) and biotinylation with biotin-DBCO (lane 5, 6). Only N₃-kethoxal labelled RNA can be biotinylated (lane 6). Bottom: Dot blot of RNA after labelling and Biotinylation reactions. Methylene blue dot results are listed as control. (e) Dot blot of isolated total RNA from mES cells which were treated by N₃-kethoxal with different periods, 1, 5, 10, 15, 20 mins. (f) Dot blot analysis of reversibility of N₃-kethoxal labelled mRNA in present of 50 mM GTP at 95° C. The N₃-kethoxal modification in mRNA was removed thoroughly after 10 mins incubation.

FIG. 2A-B. Examples of groups having chemical formula of Formula VIII (A) and kethoxal derivatives having chemical formula of Formula VI (B) are illustrated. R in FIG. 2 represent an agent coupled to the kethoxal derivative.

FIG. 3. Labeling activity of phenol-kethoxal and diphenol-kethoxal, the two compounds were incubated with a 12-mer synthetic RNA oligo containing four guanine bases, respectively. After 10 min, the reactions were cleaned-up and analyzed by MALDI-TOF.

FIG. 4. The cell permeability of phenol-kethoxal and diphenol-kethoxal was tested. Cells were treated with phenol-kethoxal and diphenol-kethoxal for 10 min, respectively, and RNA isolated from treated cells. An in vitro biotinylation reaction was performed by mixing these kethoxal derivative-labeled RNAs with biotin-phenol, horseradish peroxidase (HRP), and H₂O₂.

FIG. 5. Examples of conjugates are illustrated.

FIG. 6. Illustrates the general description of parent compound in Formula I.

FIG. 7. Illustrates non-limiting examples of Formula I.

FIG. 8A-8F. Tables illustrating various non-limiting examples of Formula I.

FIG. 9A-B. Example of LCMS results to follow relative amount of free guanosine.

### DETAILED DESCRIPTION OF THE INVENTION

Chemical labeling of nucleic acids is extremely useful for a range of applications such as probing nucleic acid structure, nucleic acid location, nucleic acid proximity information, transcription and translation. Typical labeling strategies include metabolic labeling. Coupling or tethering moieties to nucleic acids is contemplated as an anchor or tether for therapeutic or diagnostic agents to a location to which the moieties bind or associates. Certain embodiments are directed to the development of kethoxal derivatives (e.g., N₃-kethoxal) as a tethering agent.

Current methods do not specifically localize inhibitors and/or covalently lock the inhibitor in place. Embodiments described herein include an entity that localizes to a binding site and can be covalently linked at that site, e.g., tethering an inhibitory RNA to its target. Methods and compositions localize an agent to the proximity of specific target via a kethoxal derivative.

An appropriate localization signal in the form of a kethoxal derivative can be tethered to the therapeutic agent to cause it to be precisely located or fixed to or in the vicinity of its target or binding partner. Such localization anchors identify a target uniquely, or distinguish the target from a majority of incorrect targets. For example, RNA-based inhibitors of viral replication can be tethered to the target RNA. In addition, an inhibitor of a transcription complex can be locked in place altering the on/off kinetics of the inhibitor and blocking the transcription site.

Aspects include methods for enhancing the effect of a therapeutic agent in vivo. The method includes the step of causing the agent to be localized in vivo with or in the vicinity of its target.

By “enhancing” the effect of a therapeutic agent in vivo is meant that a localization anchor targets an agent to a specific site within a cell and thereby causes that agent to act more efficiently. Thus, a lower concentration of agent administered to a cell in vivo can have an equal effect to a larger concentration of non-localized agent. Such increased efficiency of the targeted or localized agent can be measured by any standard procedure well-known to those of ordinary skill in the art. In general, the effect of the agent is enhanced by placing and/or maintaining the agent in a closer proximity with the target, so that it may have its desired effect on that target.

In other aspects, the invention features methods for enhancing the effect of nucleic acid-based therapeutic agents in vivo by colocalizing or anchoring them with their target using an appropriate localization anchor.

### A. Kethoxal Derivative Anchor

Kethoxal derivative anchors enable the covalent attachment of an agent to its binding target or another entity in the vicinity. The “click” chemistry can be controlled by light, so as to achieve site-specific modification in live cells.

As described herein, N₃-kethoxal (representative of kethoxal derivatives) is shown to react selectively with guanines at single-stranded DNA and RNA. These reactions are highly efficient under mild normal cell culture conditions, and could be directly applied to tissues. Any chemical moiety can be installed on a kethoxal derivative using the methods described herein. Of particular use according to some aspects of this invention are click chemistry handles. Click chemistry handles are chemical moieties that provide a reactive group that can partake in a click chemistry reaction. Click chemistry reactions and suitable chemical groups for click chemistry reactions are well known to those of skill in the art, and include, but are not limited to terminal alkynes, azides, strained alkynes, dienes, dieneophiles, alkoxyamines, carbonyls, phosphines, hydrazides, thiols, and alkenes. For example, in some embodiments, an azide and an alkyne are used in a click chemistry reaction. In certain aspects, the “click-chemistry compatible” compounds or click chemistry handles include a terminal azide functional group (e.g., Formula I).

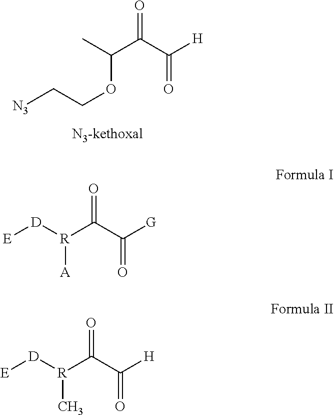

In certain aspects, compounds have a general formula of Formula I and Formula II where E is selected from a reactive group, click chemistry moiety, binding group, or therapeutic agent; D is optionally a linker or a direct bond; R is a connecting element or group; A is a substituent or a second E moiety selected independent of the first E moiety; and G is a dicarbonyl-defining group.

In certain aspects, R can be selected from substituted or unsubstituted carbon, nitrogen, aryl, alkylaryl, or heterocyclic group.

In certain aspects, A can be substituted with one or more (mono-substituted, di-substituted, etc.) of H, F, CF₃, CF₂H, CFH₂, CH₃, alkyl group, or combinations thereof. In certain aspects, A can be mono- or di-substituted with a linker. In certain aspects, A can be mono- or di-substituted with a reactive group, e.g., a click chemistry moiety, therapeutic agent, or binding moiety.

In certain aspects, D is a linker selected from an ester, amide, tetrazine, tetrazole, triazine, triazole, aryl groups, heterocycle, sulfonamide, a substituted or unsubstituted (CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. D can be —N(CH₃)—, —OCH₂—, —N(CH₃)COCH₂—, or a group having the chemical formula of Formula VII. In certain instances, the linker can be a concatamer (comprising 1, 2, 3, 4, 5, 6, 7, 8, 9, 10 or more linker(s)) of 1, 2, 3, 4, 5, 6, 7, 8, 9, 10, or more of the linkers described above.

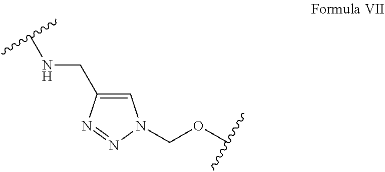 [SMILES: *NCc1cn(CO*)nn1]

In some aspects, D can be substituted with a reactive group, e.g., a click chemistry moiety. In some aspects, D can be a direct bond between E and the carbon atom binding A. In certain aspects, D can be a substituent that modulates the stability of the product formed, including alkoxy groups, ethers, carbonyls, aryl groups, electron withdrawing or electron donating groups, electrophilic of nucleophilic centers, or H-bond acceptors.

In certain aspects, G can be independently selected from H, CF₃, CF₂H, CFH₂, CH₃, or alkyl group.

In certain aspects, E can be selected from alkynes, azides, strained alkynes, dienes, dieneophiles, alkoxyamines, carbonyls, phosphines, hydrazides, thiols, alkenes, diazirines. In some aspects, E can be a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain aspects, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazrines, dienes, dieneophiles, alkoxyamines, carbonyls, phosphines, hydrazides, thiols, and alkenes. In certain aspects, E can be further coupled to an agent or binding moiety. In certain aspects the agent or binding moiety binds directly or indirectly to a target (protein or nucleic acid) in vivo, ex vivo or in vitro. In certain aspects the agent or binding moiety binds directly or indirectly to a target (protein or nucleic acid) in vivo.

In certain embodiments, kethoxal derivatives can be coupled to a variety of nucleic acids and/or small molecules (forming a kethoxal complex) that either binds and inhibits specific RNA, or to DNA or RNA reagents that bind or target RNA or DNA (such as antisense or guide RNA of CRISPR). The kethoxal component can serve to covalently lock the nucleic acid or small molecule complex. The same approach can be applied to target protein-RNA or protein-ssDNA interaction. A peptide or small molecule could bind a protein, RNA-binding protein or bind to the interface of RNA-protein interaction and the kethoxal derivative can covalently lock the inhibition.

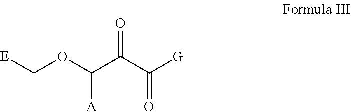

In certain aspects, N₃-kethoxal or kethoxal derivatives of Formula III or Formula IV or Formula V can be incorporated into an agent (e.g., small molecules) developed to target RNA or protein-RNA interface to enable a covalent inhibition. The kethoxal component of Formula III can react with guanines in single stranded nucleic acids to form a covalent linkage. In certain aspects the G and/or A substitution on Formula III can be independently varied to tune various properties of the kethoxal component. In certain aspects, A or G can be independently selected from H, F, CF₃, CF₂H, CFH₂, or alkyl group. For instance fluoride substitutions can be used to modulate reactivity. In certain aspects, A is a substituent or a second E moiety selected independent of the first E moiety. The modified kethoxal component could be less reactive and more specific. It could also be reversible. In certain aspects, A in Formula I, Formula III, Formula IV, Formula V, can be a substituent that modulates the stability of the product formed, selected from alkoxy groups, ethers, carbonyls, aryl groups, electron withdrawing or electron donating groups, or H-bond acceptors. The A and/or E substitutions of Formula III, Formula IV, or Formula V can be a linker that can be connected with RNA-targeting molecules. In certain aspects, the linker can be a substituent that modulates the stability of the product formed, selected from alkoxy groups, ethers, carbonyls, aryl groups, electron withdrawing or electron donating groups, or H-bond acceptors. Kethoxal derivatives can serve as a warhead to covalently lock the inhibition of the RNA-targeting molecule. “Warhead moiety” or “warhead” refers to a moiety of an inhibitor which participates, either reversibly or irreversibly, with the reaction of a donor, e.g., a protein, with a substrate. Warheads may, for example, form covalent bonds with the donor, or may create stable transition states, or be a reversible or an irreversible alkylating agent. For example, the warhead moiety can be a functional group on an inhibitor that can participate in a bond-forming reaction, wherein a new covalent bond is formed between a portion of the warhead and a donor, for example an amino acid residue of a protein. In embodiments, the warhead is an electrophile and the “donor” is a nucleophile such as the side chain of a cysteine residue. When A or E is a linker it can be connected or covalently coupled to a small molecule that binds an RNA-binding protein or binds to the interface of protein-RNA interaction. Compounds of Formula III or Formula IV or Formula V serve to covalently attached to a target (e.g., an RNA or protein) and lock the inhibition of a RNA, or a protein or protein/RNA complex. A and E can be connected to other DNA, RNA or molecules that sequence-specifically recognize RNA or ssDNA, an example is CRISPR guide RNA or any antisense developed to target RNA.

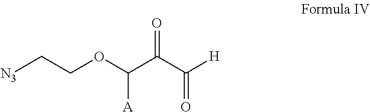

Formula IV is an example for molecules included in Formula III. The presence of N₃ makes Formula IV a candidate to be linked to fragment libraries that carry an alkyne. Formula IV can covalently target ssRNA and the N₃-alkyne click chemistry can be used to connect RNA- or protein-targeting small molecules with Formula IV. Click chemistry can be any chemical functional groups. Linker can be any and the length can be varied or adjusted. Kethoxal can be incorporated into small molecules developed to target ssDNA or protein-ssDNA interface to enable a covalent inhibition. In certain aspects, A is a substituent or a second E moiety selected independent of the first E moiety.

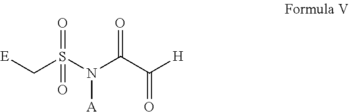

Formula V is an example for kethoxal derivative that can be rendered more electron rich and less reactive by substituting a CH₂ group with —SO₂—, in order to reduce reactivity and be potentially reversible. In certain aspects, A is a substituent or a second E moiety selected independent of the first E moiety.

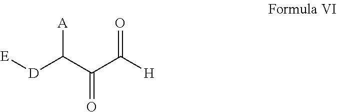

In certain aspects, a kethoxal derivative can have the general formula of Formula VI, wherein A can be hydrogen or methyl; D is optionally a linker or a direct bond; and E can be a be a reactive functional group. In certain aspects, A is a substituent or a second E moiety selected independent of the first E moiety. In some aspects, D can be a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. In some aspects, D can be substituted with a reactive group, e.g., a click chemistry moiety. In some aspects, D can be —N(CH₃)—, —OCH₂—, —N(CH₃)COCH₂—, or a group having the chemical formula of Formula VII. In certain instances, the linker can be a concatamer (comprising 1, 2, 3, 4, 5, 6, 7, 8, 9, 10 or more linker(s)) of 1, 2, 3, 4, 5, 6, 7, 8, 9, 10, or more of the linkers described above.

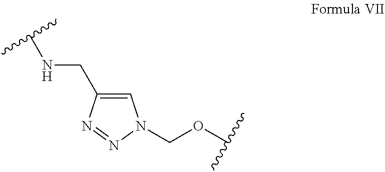 [SMILES: *NCc1cn(CO*)nn1]

In some aspects, D can be a direct bond between E and the carbon atom binding A. In some aspects, E can be substituted alkyl, heteroalkyl, substituted heteroalkyl, heteroaryl, or substituted heteroalkyl. In some aspects E can be a click chemistry moiety. In some aspects, E can be substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene.

In certain instances kethoxal derivatives are hydrated in aqueous solutions.

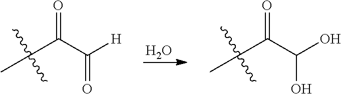

All derivatives described above may also be in hydrated forms.

In certain instances of Formulas I-VII, D, A, or A and D can be stabilization-modulating substituents. Most specifically, a H-Bond acceptor group can be added to D or A to allow it to hydrogen bond to amine-hydrogens on guanine when the kethoxal derivative reacts with guanine. With respect to A, fluoro and like groups can be used to affect reversibility.

Kethoxal derivatives fused with or further coupled with therapeutic ligands, e.g kethoxal conjugates are represented in Formula IX.

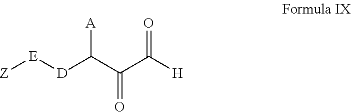

Wherein A, D and E are as defined above. In certain aspects, Z is a therapeutic agent. In some aspects, E or Z can also be any therapeutic macromolecule such as peptides, proteins, antibodies, or a ligand recognized by a therapeutic biomolecule, etc.; or a delivery vehicle such as nanoparticles, receptors, hydrogels, etc. Examples of kethoxal conjugates are illustrated in FIG. 5.

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms (C1-20 aliphatic). In certain embodiments, the aliphatic group has 1-10 carbon atoms (C1-10 aliphatic). In certain embodiments, the aliphatic group has 1-6 carbon atoms (C1-6 aliphatic). In certain embodiments, the aliphatic group has 1-5 carbon atoms (C1-5 aliphatic). In certain embodiments, the aliphatic group has 1-4 carbon atoms (C1-4 aliphatic). In certain embodiments, the aliphatic group has 1-3 carbon atoms (C1-3 aliphatic). In certain embodiments, the aliphatic group has 1-2 carbon atoms (C1-2 aliphatic). Aliphatic group substituents include, but are not limited to, any of the substituents described herein, that result in the formation of a stable moiety.

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms (C1-20alkyl). In another embodiment, the alkyl group employed contains 1-15 carbon atoms (C1-15alkyl). In another embodiment, the alkyl group employed contains 1-10 carbon atoms (C1-10alkyl). In another embodiment, the alkyl group employed contains 1-8 carbon atoms (C1-8alkyl). In another embodiment, the alkyl group employed contains 1-6 carbon atoms (C1-6alkyl). In another embodiment, the alkyl group employed contains 1-5 carbon atoms (C1-5alkyl). In another embodiment, the alkyl group employed contains 1-4 carbon atoms (C1-4alkyl). In another embodiment, the alkyl group employed contains 1-3 carbon atoms (C1-3alkyl). In another embodiment, the alkyl group employed contains 1-2 carbon atoms (C1-2alkyl). Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents. Alkyl group substituents include, but are not limited to, any of the substituents described herein, that result in the formation of a stable moiety.

The term “alkylaryl” refers to a radical containing both aliphatic and aromatic structures, an aryl group bonded directly to an alkyl group.

The term “alkylene,” as used herein, refers to a biradical derived from an alkyl group, as defined herein, by removal of two hydrogen atoms. Alkylene groups may be cyclic or acyclic, branched or unbranched, substituted or unsubstituted. Alkylene group substituents include, but are not limited to, any of the substituents described herein, that result in the formation of a stable moiety.

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms (C2-20alkenyl). In some embodiments, the alkenyl group employed in the invention contains 2-15 carbon atoms (C2-15alkenyl). In another embodiment, the alkenyl group employed contains 2-10 carbon atoms (C2-10alkenyl). In still other embodiments, the alkenyl group contains 2-8 carbon atoms (C2-8alkenyl). In yet other embodiments, the alkenyl group contains 2-6 carbons (C2-6alkenyl). In yet other embodiments, the alkenyl group contains 2-5 carbons (C2-5alkenyl). In yet other embodiments, the alkenyl group contains 2-4 carbons (C2-4alkenyl). In yet other embodiments, the alkenyl group contains 2-3 carbons (C2-3alkenyl). In yet other embodiments, the alkenyl group contains 2 carbons (C2alkenyl). Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety. The term “alkenylene,” as used herein, refers to a biradical derived from an alkenyl group, as defined herein, by removal of two hydrogen atoms. Alkenylene groups may be cyclic or acyclic, branched or unbranched, substituted or unsubstituted. Alkenylene group substituents include, but are not limited to, any of the substituents described herein, that result in the formation of a stable moiety.

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms (C2-20alkynyl). In some embodiments, the alkynyl group employed in the invention contains 2-15 carbon atoms (C2-15alkynyl). In another embodiment, the alkynyl group employed contains 2-10 carbon atoms (C2-10alkynyl). In still other embodiments, the alkynyl group contains 2-8 carbon atoms (C2-8alkynyl). In still other embodiments, the alkynyl group contains 2-6 carbon atoms (C2-6alkynyl). In still other embodiments, the alkynyl group contains 2-5 carbon atoms (C2-5alkynyl). In still other embodiments, the alkynyl group contains 2-4 carbon atoms (C2-4alkynyl). In still other embodiments, the alkynyl group contains 2-3 carbon atoms (C2-3alkynyl). In still other embodiments, the alkynyl group contains 2 carbon atoms (C2alkynyl). Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety. The term “alkynylene,” as used herein, refers to a biradical derived from an alkynylene group, as defined herein, by removal of two hydrogen atoms. Alkynylene groups may be cyclic or acyclic, branched or unbranched, substituted or unsubstituted. Alkynylene group substituents include, but are not limited to, any of the substituents described herein, that result in the formation of a stable moiety.

The term “carbocyclic” or “carbocyclyl” as used herein, refers to an as used herein, refers to a cyclic aliphatic group containing 3-10 carbon ring atoms (C3-10carbocyclic). Carbocyclic group substituents include, but are not limited to, any of the substituents described herein, that result in the formation of a stable moiety.

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that further contains one or more heteroatoms (e.g., oxygen, sulfur, nitrogen, phosphorus, or silicon atoms) between carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl,” “heteroalkynyl,” and the like. Furthermore, as used herein, the terms “heteroalkyl,” “heteroalkenyl,” “heteroalkynyl,” and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms and 1-6 heteroatoms (C1-20heteroaliphatic). In certain embodiments, the heteroaliphatic group contains 1-10 carbon atoms and 1-4 heteroatoms (C1-10heteroaliphatic). In certain embodiments, the heteroaliphatic group contains 1-6 carbon atoms and 1-3 heteroatoms (C1-6heteroaliphatic). In certain embodiments, the heteroaliphatic group contains 1-5 carbon atoms and 1-3 heteroatoms (C1-5heteroaliphatic). In certain embodiments, the heteroaliphatic group contains 1˜4 carbon atoms and 1-2 heteroatoms (C1-4heteroaliphatic). In certain embodiments, the heteroaliphatic group contains 1-3 carbon atoms and 1 heteroatom (C1-3heteroaliphatic). In certain embodiments, the heteroaliphatic group contains 1-2 carbon atoms and 1 heteroatom (C1-2heteroaliphatic). Heteroaliphatic group substituents include, but are not limited to, any of the substituents described herein, that result in the formation of a stable moiety.

The term “heteroalkyl,” as used herein, refers to an alkyl moiety, as defined herein, which contain one or more heteroatoms (e.g., oxygen, sulfur, nitrogen, phosphorus, or silicon atoms) in between carbon atoms. In certain embodiments, the heteroalkyl group contains 1-20 carbon atoms and 1-6 heteroatoms (C1-20 heteroalkyl). In certain embodiments, the heteroalkyl group contains 1-10 carbon atoms and 1-4 heteroatoms (C1-10 heteroalkyl). In certain embodiments, the heteroalkyl group contains 1-6 carbon atoms and 1-3 heteroatoms (C1-6 heteroalkyl). In certain embodiments, the heteroalkyl group contains 1-5 carbon atoms and 1-3 heteroatoms (C1-5 heteroalkyl). In certain embodiments, the heteroalkyl group contains 1-4 carbon atoms and 1-2 heteroatoms (C1-4 heteroalkyl). In certain embodiments, the heteroalkyl group contains 1-3 carbon atoms and 1 heteroatom (C1-3 heteroalkyl). In certain embodiments, the heteroalkyl group contains 1-2 carbon atoms and 1 heteroatom (C1-2 heteroalkyl). The term “heteroalkylene,” as used herein, refers to a biradical derived from an heteroalkyl group, as defined herein, by removal of two hydrogen atoms. Heteroalkylene groups may be cyclic or acyclic, branched or unbranched, substituted or unsubstituted. Heteroalkylene group substituents include, but are not limited to, any of the substituents described herein, that result in the formation of a stable moiety.

The term “heteroalkenyl,” as used herein, refers to an alkenyl moiety, as defined herein, which further contains one or more heteroatoms (e.g., oxygen, sulfur, nitrogen, phosphorus, or silicon atoms) in between carbon atoms. In certain embodiments, the heteroalkenyl group contains 2-20 carbon atoms and 1-6 heteroatoms (C2-20 heteroalkenyl). In certain embodiments, the heteroalkenyl group contains 2-10 carbon atoms and 1-4 heteroatoms (C2-10 heteroalkenyl). In certain embodiments, the heteroalkenyl group contains 2-6 carbon atoms and 1-3 heteroatoms (C2-6 heteroalkenyl). In certain embodiments, the heteroalkenyl group contains 2-5 carbon atoms and 1-3 heteroatoms (C2-5 heteroalkenyl). In certain embodiments, the heteroalkenyl group contains 2-4 carbon atoms and 1-2 heteroatoms (C2-4 heteroalkenyl). In certain embodiments, the heteroalkenyl group contains 2-3 carbon atoms and 1 heteroatom (C2-3 heteroalkenyl). The term “heteroalkenylene,” as used herein, refers to a biradical derived from an heteroalkenyl group, as defined herein, by removal of two hydrogen atoms. Heteroalkenylene groups may be cyclic or acyclic, branched or unbranched, substituted or unsubstituted.

The term “heteroalkynyl,” as used herein, refers to an alkynyl moiety, as defined herein, which further contains one or more heteroatoms (e.g., oxygen, sulfur, nitrogen, phosphorus, or silicon atoms) in between carbon atoms. In certain embodiments, the heteroalkynyl group contains 2-20 carbon atoms and 1-6 heteroatoms (C2-20 heteroalkynyl). In certain embodiments, the heteroalkynyl group contains 2-10 carbon atoms and 1-4 heteroatoms (C2-10 heteroalkynyl). In certain embodiments, the heteroalkynyl group contains 2-6 carbon atoms and 1-3 heteroatoms (C2-6 heteroalkynyl). In certain embodiments, the heteroalkynyl group contains 2-5 carbon atoms and 1-3 heteroatoms (C2-5 heteroalkynyl). In certain embodiments, the heteroalkynyl group contains 2-4 carbon atoms and 1-2 heteroatoms (C2-4 heteroalkynyl). In certain embodiments, the heteroalkynyl group contains 2-3 carbon atoms and 1 heteroatom (C2-3 heteroalkynyl). The term “heteroalkynylene,” as used herein, refers to a biradical derived from an heteroalkynyl group, as defined herein, by removal of two hydrogen atoms. Heteroalkynylene groups may be cyclic or acyclic, branched or unbranched, substituted or unsubstituted.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety.

The term “aryl,” as used herein, refers to an aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C4-C20 aromatic ring system having one, two, or three aromatic rings which include, but are not limited to, phenyl, biphenyl, naphthyl, and the like, which may bear one or more substituents. Aryl substituents include, but are not limited to, any of the substituents described herein, that result in the formation of a stable moiety. The term “arylene,” as used herein refers to an aryl biradical derived from an aryl group, as defined herein, by removal of two hydrogen atoms. Arylene groups may be substituted or unsubstituted. Arylene group substituents include, but are not limited to, any of the substituents described herein, that result in the formation of a stable moiety. Additionally, arylene groups may be incorporated as a linker group into an alkylene, alkenylene, alkynylene, heteroalkylene, heteroalkenylene, or heteroalkynylene group, as defined herein.

The term “heteroaryl,” as used herein, refers to an aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Examples of heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety. The term “heteroarylene,” as used herein, refers to a biradical derived from an heteroaryl group, as defined herein, by removal of two hydrogen atoms. Heteroarylene groups may be substituted or unsubstituted.

Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety.

The term “acyl,” as used herein, is a subset of a substituted alkyl group, and refers to a group having the general formula —C(═O)RA, —C(═O)ORA, —C(═O)—O—C(═O)RA, —C(═O)SRA, —C(═O)N(RA)₂, —C(═S)RA, —C(═S)N(RA)₂, and —C(═S)S(RA), —C(═NRA)RA, —C(═NRA)ORA, —C(═NRA)SRA, and —C(═NRA)N(RA)₂, wherein RA is hydrogen; halogen; substituted or unsubstituted hydroxyl; substituted or unsubstituted thiol; substituted or unsubstituted amino; acyl; optionally substituted aliphatic; optionally substituted heteroaliphatic; optionally substituted alkyl; optionally substituted alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di heteroarylamino; or two RA groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety.

The term “acylene,” as used herein, is a subset of a substituted alkylene, substituted alkenylene, substituted alkynylene, substituted heteroalkylene, substituted heteroalkenylene, or substituted heteroalkynylene group, and refers to an acyl group having the general formulae: R₀—(C═X₁)—R₀—, —R—X₂(C═X₁)—R₀—, or —R₀—X₂(C═X₁)X₃—R₀—, where X₁, X₂, and X₃ is, independently, oxygen, sulfur, or NRr, wherein Rr is hydrogen or optionally substituted aliphatic, and R₀ is an optionally substituted alkylene, alkenylene, alkynylene, heteroalkylene, heteroalkenylene, or heteroalkynylene group, as defined herein. Exemplary acylene groups wherein R₀ is alkylene includes —(CH₂)T-O(C═O)—(CH₂)T-; (CH₂)T-NRr(C═O)—(CH₂)T-; —(CH₂)T-O(C=NRr)-(CH₂)T-; —(CH₂)T-NRr(C=NRr)-(CH₂)T-; —(CH₂)T-(C═O)—(CH₂)T-; —(CH₂)T-(C=NRr)-(CH₂)T-; —(CH₂)T-S(C═S)—(CH₂)T-; —(CH₂)T-NRr(C═S)—(CH₂)—; —(CH₂)T-S(C=NRr)-(CH₂)T-; —(CH₂)T-O(C═S)—(CH₂)T-; —(CH₂)T-(C═S)—(CH₂)T-; or —(CH₂)T-S(C═O)—(CH₂)T-, and the like, which may bear one or more substituents; and wherein each instance of T is, independently, an integer between 0 to 20. Acylene substituents include, but are not limited to, any of the substituents described herein, that result in the formation of a stable moiety.

The term “amino,” as used herein, refers to a group of the formula (—NH₂). A “substituted amino” refers either to a mono-substituted amine (—NHRh) of a disubstituted amine (—NRh₂), wherein the Rh substituent is any substituent as described herein that results in the formation of a stable moiety (e.g., an amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the Rh substituents of the di-substituted amino group (—NRh₂) form a 5- to 6-membered heterocyclic ring.

The term “hydroxy” or “hydroxyl,” as used herein, refers to a group of the formula (—OH). A “substituted hydroxyl” refers to a group of the formula (—ORO, wherein Ri can be any substituent which results in a stable moiety (e.g., a hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “thio” or “thiol,” as used herein, refers to a group of the formula (—SH). A “substituted thiol” refers to a group of the formula (—SRr), wherein Rr can be any substituent that results in the formation of a stable moiety (e.g., a thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “imino,” as used herein, refers to a group of the formula (=NRr), wherein Rr corresponds to hydrogen or any substituent as described herein, that results in the formation of a stable moiety (for example, an amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted).

The term “azide” or “azido,” as used herein, refers to a group of the formula (—N₃).

The terms “halo” and “halogen,” as used herein, refer to an atom selected from fluorine (fluoro, —F), chlorine (chloro, —Cl), bromine (bromo, —Br), and iodine (iodo, —I).

B. Synthesis of Kethoxal Derivatives.

Kethoxal and its analogs were first reported to react with and inactivate the RNA virus since the 1950s (Staehelin, Biochimca Biophysica Acta 31:448-54, 1959). The 1,2-dicarbonyl group of kethoxal showed high specificity to guanine, which make it very useful in the probing of RNA secondary structure. In addition, other kethoxal derivatives, such as kethoxal bis(thiosemicarbazone)(KTS)(Booth and Sartorelli, Nature 210:104-5, 1966) displayed promising anticancer activity, bikethoxal (Brewer et al., Biochemistry 22:4303-9, 1983) demonstrated the ability to cross-link RNA and proteins within intact ribosomal 30S and 505 subunits. However, it is surprising that the synthesis of kethoxal and its derivatives are rarely reported. A review of the literature indicates that kethoxal preparation was mostly based on oxidation by selenium dioxide following purification by vacuum distillation (Brewer et al., Biochemistry 22:4303-9, 1983; Tiffany et al., Journal of the American Chemical Society 79:1682-87, 1957; Lo et al., Journal of Labelled Compounds and Radiopharmaceuticals 44:S654-S656, 2001). This method has several limitations. First, metal oxidation reaction always results in byproducts. Second, the excess selenium was hard to remove. Third, synthesis of kethoxal derivatives with other functional groups is difficult because the reagents with functional groups may not survive with selenium dioxide under reflux conditions. For example, studies indicate that azide- and thiol-modified kethoxal cannot be prepared by selenium dioxide oxidation. Lastly, vacuum distillation purification is not suitable for kethoxal derivatives with high-molecular weight.

Glyoxal and its analogs are sensitive to air and therefore cannot be purified by chromatography (Jiang et al., Organic Letters 3:4011-13, 2001). The mild oxidation of diazoketone by freshly prepared dimethyl-dioxirane (DMD) can produce a glyoxal functional group in quantitative yield (Jiang et al., Organic Letters 3:4011-13, 2001). In this study, azide-kethoxal was prepared through a novel synthetic strategy following a three-step synthesis (Scheme S1). The advantage of the synthetic process is its easy-to-operate and is high yield. What's more, this strategy is also convenient for the preparation of other kethoxal derivatives with various functional groups.

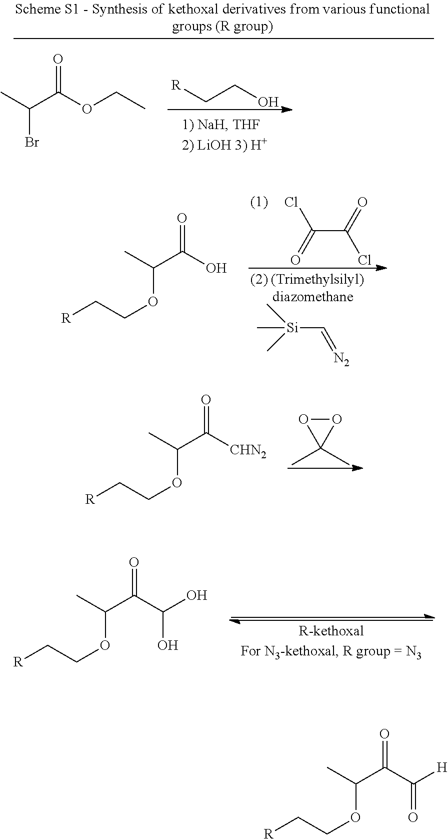

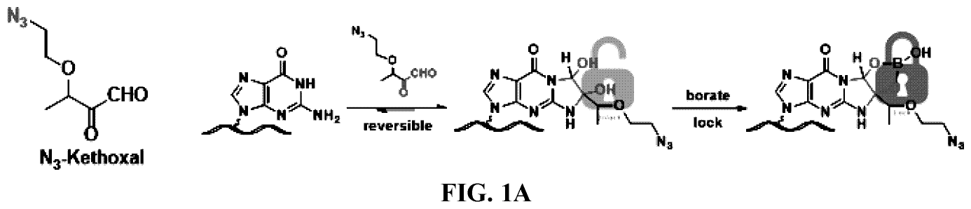

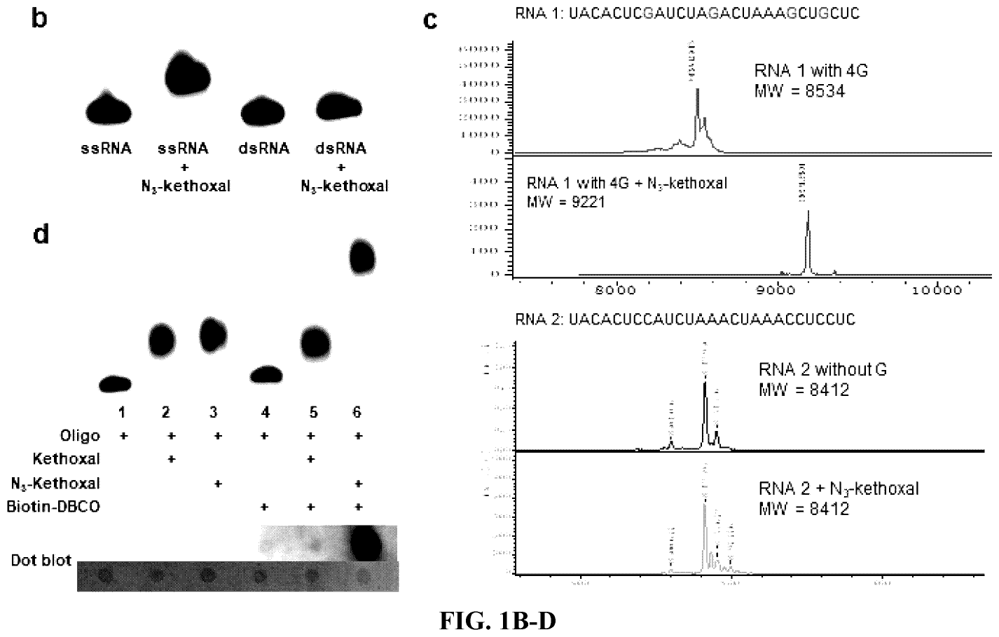

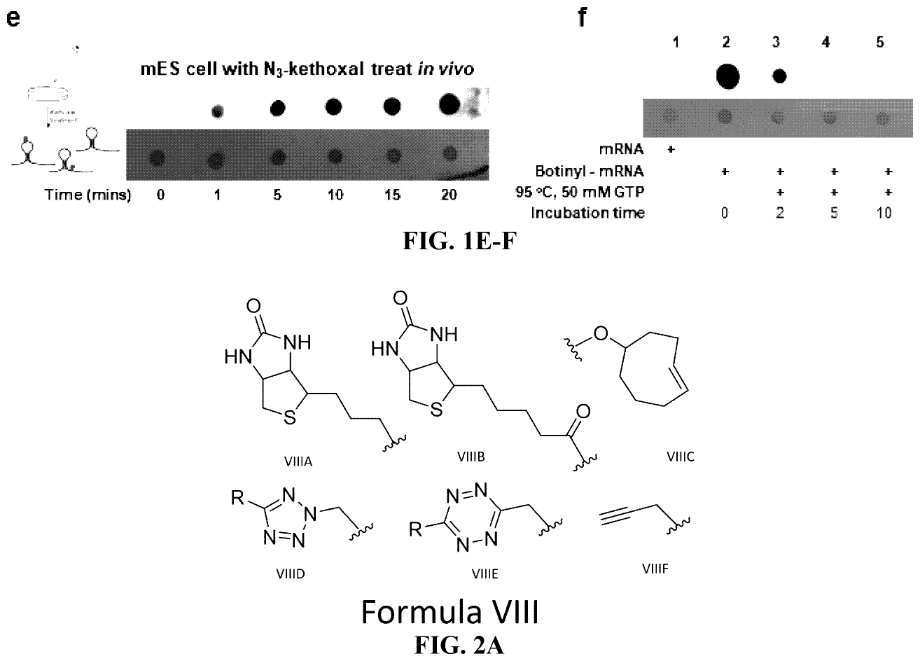

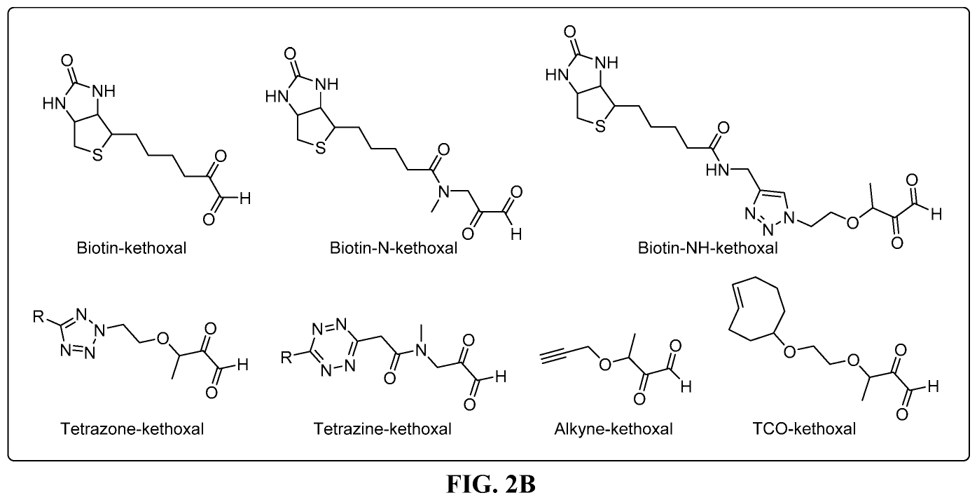

N₃-kethoxal reacts with guanines in single-stranded DNA and RNA. Kethoxal (1,1-dihydroxy-3-ethoxy-2-butanone), is known to react with guanines specifically at N₁ and N₂ position at the Watson-Crick interface (Shapiro et al., Biochemistry 8:238-45, 1969). Due to challenges in synthesis, kethoxal has not been further functionalized and widely applied to nucleic acid labeling previously. Described herein is the development of N₃-kethoxal (FIG. 1a), which not only inherits the reactivity towards guanines from its parent molecule, but also contains an azido group, which serves as a bio-orthogonal handle to be further functionalized through ‘click’ chemistry. With MALDI-TOF analysis, it was shown that N₃-kethoxal efficiently labels guanines on RNA, while no reactivity was observed on other bases. It was further demonstrated the selectivity of N₃-kethoxal on single-stranded DNA/RNA by using gel electrophoresis. After incubation with N₃-kethoxal, a shift was observed on single-stranded RNA on the gel, indicating the formation of the RNA-kethoxal complex, while no such shift was detected with double-stranded RNA. It was also shown that N₃-kethoxal is highly cell-permeable and can label DNA and RNA in living cells within 5 min, which makes it suitable for further applications.

C. Single-Stranded DNA Mapping (ssDNA-seq)

Kethoxal derivatives of the present invention enables genome-wide single-stranded DNA mapping (ssDNA-seq). Taking advantage of the sensitivity and the selectivity of kethoxal derivatives towards single-stranded nucleic acids, kethoxal derivatives were first applied to map single-stranded regions of the genome, which has not been previously achieved. One procedure for ssDNA mapping can comprise one or more of the following steps. First step can be preparing a labeling medium by adding a kethoxal derivative to a cell culture medium. Incubating cells in the labeling medium for a desired time, at a desired temperature, under desired conditions. Transcription inhibition studies can be performed by treating cells under DRB or triptolide or equivalent reagent prior to incubating in kethoxal derivative-containing medium. After incubation, harvesting the cells, and isolating total DNA from the cells. DNA can be suspended in FhO and in the presence of DBCO-PEG4-biotin (DMSO solution) and incubated at an appropriate temperature for an appropriate time, e.g., 37° C. for 2 h. RNase A can be added to the reaction mixture and the mixture incubated for an appropriate time at an appropriate temperature, e.g., 37° C. for 15 min. 7. DNA can be recovered from the reaction mixture and used to construct libraries. Libraries can be constructed using various commercial library construction kits, for example Accel-NGS Methyl-seq DNA library kit (Swift) or Kapa Hyper Plus kit (Kapa Biosystems). The next step can include sequencing libraries, for example on a Nextseq SR80 mode and perform downstream analysis.

D. Kethoxal-Assisted RNA-RNA Interaction Mapping (KARRI)

Considering the reactivity of kethoxal derivatives towards RNA, kethoxal-assisted RNA-RNA interaction mapping (KARRI) was developed based on kethoxal derivative labeling and dendrimer crosslinking of interacting RNA-RNA. To demonstrate KARRI mapping, formaldehyde-fixed mouse embryonic stem cells (mESC) were treated with kethoxal derivative and then incubated with PAMAM dendrimers (Esfand and Tomalia, (2001) Drug Discov. Today 6:427-36) decorated with two dibenzocyclooctyne (DBCO) molecules and one biotin molecule at the surface. Each PAMAM dendrimer chemically crosslinks two proximal kethoxal derivative labeled guanines through the “click” reaction, and provides a handle for enrichment through the biotin moiety on it. After crosslinking, RNAs were isolated, fragmented and subjected to immunoprecipitation by streptavidin beads. Proximity ligation was then performed on beads and the product RNA was used for library construction. Sequencing reads were aligned with only chimeric reads used for RNA-RNA interaction analysis.

Procedure for kethoxal-Assisted RNA-RNA interaction (KARRI). The KARRI methods can include one or more of the following steps. Cells can be suspended in a fixative, e.g., formaldehyde solution, and incubated at room temperature with gentle rotate. The reaction can be quenched, e.g., by adding glycine. For translation inhibitor treatment, cells are treated with cycloheximide or harringtonine. Cells are collected and aliquoted. Kethoxal derivative can be diluted 1:5 using an appropriate solvent, e.g., DMSO, and incorporated into a labeling buffer (kethoxal derivative, lysis buffer (10 mM Tris-HCl pH 8.0, 10 mM NaCl, 0.2 IGEPAL CA630) and proteinase inhibitor cocktail). Cells can be suspended in labeling buffer and cells collected after incubation. Collected cells can be washed in ice-cold lysis buffer 1, 2,3 or more times. The cell pellet can be suspended in MeOH containing cross-linkers and the cells collected. RNA can be extracted and purified. RNA pellets can be suspended in H2O, with DNase I buffer (100 mM Tris-HCl pH 7.4, 25 mM MgCl₂, 1 mM CaCl₂), DNase I, RNase inhibitor, and incubated with gentle shaking. The mixture is then exposed to proteinase K. RNA is extracted with phenol-chloroform and purified RNA by EtOH precipitation. RNA pellets are suspended in H₂O and fragmentation buffer with RNase inhibitor and incubated. Fragmentation is stopped by additional of fragmentation stop buffer and the sample is put on ice to quench the reaction. Crosslinked RNA is enriched by using pre-washed Streptavidin beads. Beads are mixed with DNA and the mixture was incubated at room temperature with gentle rotate. After incubation, beads were washed. Washed beads are suspended in H₂O with PNK buffer and T4 PNK, RNase inhibitor and shaken for a first incubation period, then another aliquot of T4 PNK and ATP are added and shaken for a second incubation period. Beads are washed and suspended in a ligase solution. After incubation in ligase solution the beads are washed. RNA is eluted by heating and the RNA recovered. Half of the recovered RNA is used for library construction. Libraries are sequenced and downstream analysis performed.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of Kethoxal Derivatives

The synthesis route of N₃-kethoxal.

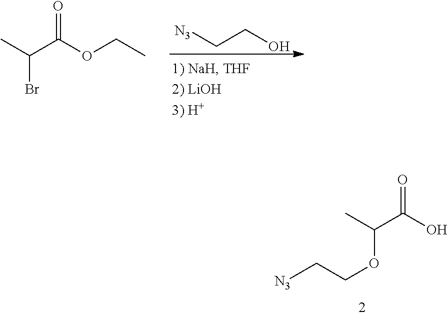

2-(2-azidoethoxy)propanoic acid 2: Sodium hydride (60% dispersion in mineral oil, 6 g, 0.15 mol) was added to a 250 mL two-necked flask, then anhydrous THF 50 mL was added under N₂ condition. The suspension was vigorously stirred and cooled to 0° C. 2-Azidoenthanol (8.7 g, 0.1 mol) in 20 mL anhydrous THF was added dropwise over 20 minutes. The solution was stirred at an ambient temperature for 15 mins, then cooled to 0° C. again. Ethyl 2-bromopropionate (27.15 g, 0.15 mol) in 10 mL THF was added dropwise. The reaction mixture was warmed to room temperature and stirred overnight under N₂ atmosphere. 100 mL Water was used to quench the reaction and the resulted mixture was washed by diethyl ether three times (3×100 mL). The combined organic layers were dried over anhydrous Na₂SO₄. The crude product was dissolved in 50 ml THF and was added to LiOH aqueous solution (40 ml, 1 M). The mixture was stirred for 16 h at room temperature. THF was removed and HCl (2 M) was added to pH 2. Then, the THF was extracted by diethyl ether three times (3×100 ml). The combined organic layers were dried over anhydrous NaSO₄. After concentration and silica gel chromatography (ethyl acetate:petroleum ether=1:7), the product 2 was collected as colorless oil (6.67 g, 26%). ¹H NMR (400 MHz, CDCl₃): δ=4.09 (q, J=6.9 Hz, 1H), 3.85 (ddd, J=9.8, 5.9, 3.4 Hz, 1H), 3.66-3.58 (m, 1H), 3.55-3.46 (m, 1H), 3.42-3.33 (m, 1H), 1.49 (t, J=9.4 Hz, 3H). ¹³C NMR (101 MHz, CDCl₃): δ=178.48, 74.98, 69.13, 50.65, 18.47. HRMS C₃H₉N₃O₃⁺ [M+H]⁺ calculated 160.07167, found 160.07091.

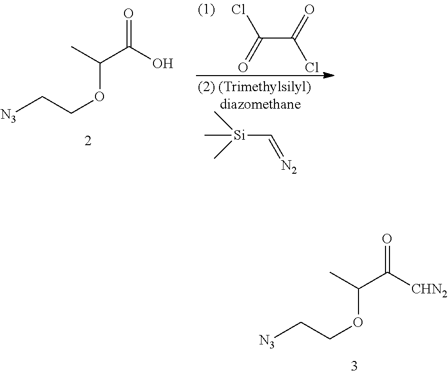

3-(2-azidoethoxy)-1-diazopentane-2-one 3: Under N₂ condition, 2 (1.59 g, 10 mmol) was dissolved in 15 mL anhydrous CH₂C12 and one drop of DMF. Oxalyl chloride (926 μL, 15 mmol) was added to the solution and stirred at room temperature for 2 h. After that, the solvent and excess oxalyl chloride was removed. The residue was dissolved in anhydrous CH₃CN 50 mL, cooled to 0° C., and (Trimethylsilyl)diazomethane solution 2 M in diethyl ether (4 mL, 10 mmol) was added dropwise. The reaction mixture was stirred at 0° C. overnight. The solvent was evaporated and silica gel chromatography (ethyl acetate:petroleum ether=1:7) was performed in order to afford product 3 as yellow oil (620 mg, 33.8%). ¹H NMR (400 MHz, CDCl₃): δ=5.82 (s, 1H), 4.00-3.85 (m, 1H), 3.72-3.60 (m, 2H), 3.48-3.35 (m, 2H), 1.38 (d, J=6.8 Hz, 3H). ¹³C NMR (101 MHz, CDCl₃): δ=196.94, 80.89, 68.73, 52.30, 50.88, 18.58. HRMS C₆H₉N₅O₂⁺ [M+H]⁺ calculated 184.0829, found 184.0822.

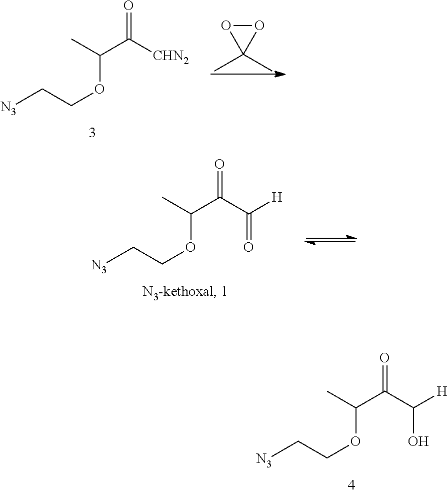

Azido-kethoxal 1 (N₃-kethoxal), or 3-(2-azidoethoxy)-1,1-dihydroxybutan-2-one (4):

According to Adam's procedure, the Dimethyldioxirane (DMD) in an acetone solution was prepared. To the compound 3 (183 mg, 1 mmol), 11 mL DMD-acetone was added in several portions. Obvious gas evolution was observed. The reaction mixture was stirred at room temperature until the reaction was complete under TLC monitor to Azido-kethoxal 1 and its hydyate 4 as a yellow oil. ¹H NMR (400 MHz, CDCl₃): δ=[9.5 (m)+5.5 (m), 1H], 4.55-4.40 (m, 1H), 3.75 (m, 2H), 3.50-3.25 (m, 2H), 1.50-1.20 (m, 3H). HRMS C₆H₉N₃O₃⁺ [M+Na]⁺ calculated 194.0536, found 194.0555.

General chemical and biological materials. All chemical reagents for N₃-kethoxal synthesis were purchased from commercial sources. RNA oligoes were purchased from Integrated DNA Technologies, Inc. (IDT) and Takara Biomedical Technology Co., Ltd. Buffer salts and chemical reagents for N₃-kethoxal synthesis were purchased from commercial sources. Superscript III, Dynabeads® MyOne™ Streptavidin C1 was purchased from Life technologies. T4 PNK, T4 RNL2tr K227Q, 5′-Deadenylase, RecJ_{f} were purchased from New England Biolabs. CircLigaseII was purchase from epicenter company. DBCO-Biotin was purchase from Click Chemistry Tools LLC (A116-10). All RNase-free solutions were prepared from DEPC-treated MilliQ-water.

### Synthesis Scheme of Carbon-Kethoxal (5-azido-2-oxopentanal)

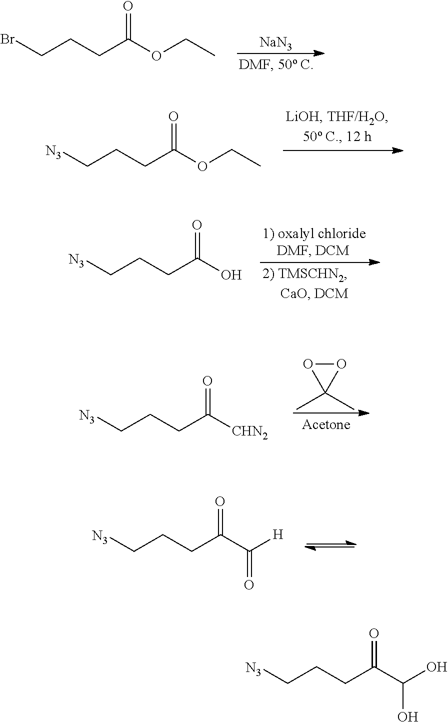

Synthetic Route for carbon-kethoxal (5-azido-2-oxopentanal). Ethyl 4-azidobutyrate: A solution of ethyl 4-bromobutyrate (7.802 g, 40 mmol), NaN₃ (3.900 g, 60 mmol, 15 equiv.) and 6 ml of water in 18 ml of acetone was refluxed for 5 h. After the reaction finished, the acetone was removed by vacuum and residue was partitioned between Et₂O (200 ml) and water (100 ml). The organic layer was separated, and the water layer was extracted with 200 mL Et₂O, twice. The combined organic layer was washed with water followed by drying over anhydrous Na₂SO₄. After filtration and evaporation of the solvent, silica gel chromatography was performed (ethyl acetate:petroleum ether=1:50) and ethyl 4-azidobutyrate (6.21 g, quant.) was obtained as a colorless oil. ¹H NMR (400 MHz, CDCl₃) δ 4.05 (q, J=7.2 Hz, 2H), 3.39 (t, J=6.5 Hz, 2H), 2.40 (t, J=7.2 Hz, 2H), 2.08 (p, J=6.7 Hz, 2H), 1.18 (t, J=7.2 Hz, 3H).

4-azidobutanoic acid: The above product ethyl 4-azidobutyrate (2.583 g, 20 mmol) was suspended in a mixture of LiOH.H₂O (2.520 g, 60 mmol, 3.0 eq) in water (30 mL) and THF (10 mL). The mixture was stirred at 50° C. for 12 h. THF was removed and HCl (2 M) was added to adjust pH to 2. Then, the THF was extracted by diethyl ether three times (3×100 ml). The combined organic layers were dried over anhydrous NaSO₄. After concentration and silica gel chromatography (acetone:petroleum ether=1:10 to 1:2), the product 4-azidobutanoic acid was collected as colorless oil (2.011 g, 78%). ¹H NMR (400 MHz, CDCl₃) δ 10.19 (s, 1H), 3.36 (t, J=6.7 Hz, 2H), 2.46 (t, J=7.2 Hz, 2H), 1.90 (p, J=6.9 Hz, 2H).

5-azido-1-diazopentan-2-one: Under inert conditions (N₂), the above product 4-azidobutanoic acid (646 mg, 5 mmol) was dissolved in 15 mL anhydrous CH₂C12 and chilled at 0° C. DMF and oxalyl chloride (650 μL, 7.5 mmol) were added to the solution dropwise. After warming the reaction mixture to room temperature, it was stirred for 2 h. After that, the solvent and excess oxalyl chloride were removed. The residue was dissolved in anhydrous CH₂Cl₂ 25 mL, cooled to 0° C., and CaO (308 mg, 5.5 mmol, 1.1 equiv.) was added. To this, 2M TMSCHN₂ solution in diethyl ether (2.5 mL, 5 mmol) was added dropwise. The reaction mixture was stirred at 0° C. overnight. The solvent was evaporated and silica gel chromatography (ethyl acetate:petroleum ether=1:5) was performed in order to afford product 5-azido-1-diazopentan-2-one as yellow oil (680 mg, 89%). ¹H NMR (400 MHz, CDCl₃) δ 5.30 (s, 1H), 3.35 (t, J=6.6 Hz, 2H), 2.42 (s, 2H), 1.92 (p, J=6.9 Hz, 2H).

Carbon kethoxal (5-azido-2-oxopentanal): According to Adam's procedure, the dimethyldioxirane (DMD) in an acetone solution was prepared. To 5-azido-1-diazopentan-2-one (39 mg, 0.28 mmol), 5 mL DMD-acetone was added and gas evolution was observed. The reaction mixture was stirred at room temperature until the reaction was completed (under TLC monitoring) to form carbon kethoxal and its hydrate as a yellow oil (quant.). ¹H NMR (400 MHz, CDCl₃): δ=[9.23 (m)+5.24 (m), 1H], 3.41-3.31 (m, 2H), 3.01-2.46 (m, 2H), 1.96-1.80 (m, 2H).

### Synthetic Scheme for Mono-Fluoride Kethoxal (3-(2-azidoethoxy)-3-fluoro-2-oxopropanal)

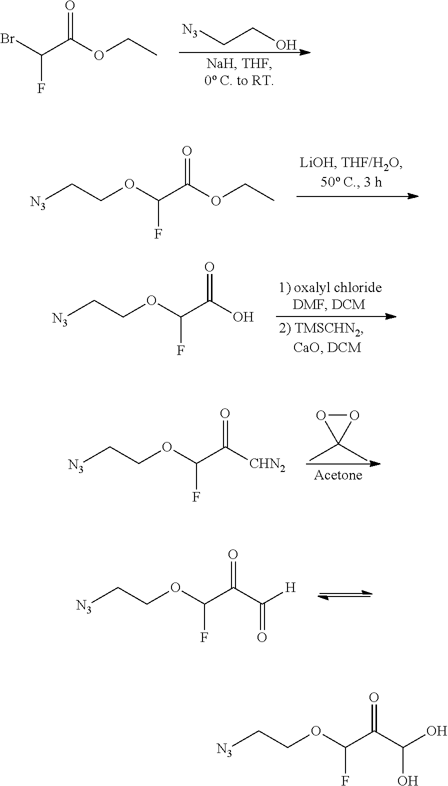

Synthetic Route for mono-fluoride kethoxal (3-(2-azidoethoxy)-3-fluoro-2-oxopropanal): ethyl 2-(2-azidoethoxy)-2-fluoroacetate:Sodium hydride (4.4 g) was added to anhydrous THF. The suspension was vigorously stirred and cooled to 0° C. 2-azidoenthanol (6.416 g) in 20 mL anhydrous THF was added dropwise. The solution was stirred at RT for 15 min, then cooled to 0° C. again. Ethyl 2-bromopropionate (14.868 g) in 10 mL THF was added dropwise. The reaction mixture was warmed to room temperature and stirred overnight. Water was used to quench the reaction, followed by extraction with diethyl ether. The combined organic layers were dried over anhydrous Na₂SO₄. After filtration and evaporation of solvent, silica gel chromatography was performed (ethyl acetate:petroleum ether=1:50 to 1:30), and ethyl 2-(2-azidoethoxy)-2-fluoroacetate (8.832 g, 64%) was obtained as a colorless oil.

2-(2-azidoethoxy)-2-fluoroacetic acid: The above product ethyl 2-(2-azidoethoxy)-2-fluoroacetate (7.5 g) was suspended in a mixture of LiOH.H₂O (4.93 g) in water and THF. The mixture was stirred at 50° C. for 3 h. THF was removed and HCl (2 M) was added to adjust the mixture to pH 2. The THF was next extracted by diethyl ether. The combined organic layers were dried over anhydrous NaSO₄. After concentration and silica gel chromatography (acetone:petroleum ether=1:10 to 1:5), the product 2-(2-azidoethoxy)-2-fluoroacetic acid was collected as colorless oil (3.80 g, 60%).

1-(2-azidoethoxy)-3-diazo-1-fluoropropan-2-one: Under inert conditions (N₂), the above product 2-(2-azidoethoxy)-2-fluoroacetic acid (200 mg) was dissolved in anhydrous CH₂C12 and chilled to 0° C. DMF and oxalyl chloride (158 μL) was added to the solution dropwise. After warming the reaction mixture to room temperature, it was stirred for 2 h. The solvent and excess oxalyl chloride were removed. The residue was dissolved in anhydrous CH₂C12, cooled to 0° C., and CaO (76 mg) was added. A 2M TMSCHN₂ solution in diethyl ether (0.31 mL) was added dropwise to the mixture and was stirred at 0° C. overnight. The solvent was evaporated and silica gel chromatography (ethyl acetate:petroleum ether=1:20 to 1:5) was performed in order to afford the product 1-(2-azidoethoxy)-3-diazo-1-fluoropropan-2-one as yellow oil (180 mg, 79%).

Mono-fluoride kethoxal (3-(2-azidoethoxy)-3-fluoro-2-oxopropanal): According to Adam's procedure, the dimethyldioxirane (DMD) in an acetone solution was prepared. To 1-(2-azidoethoxy)-3-diazo-1-fluoropropan-2-one (47 mg), DMD-acetone was added, and obvious gas evolution was observed. The reaction mixture was stirred at room temperature until the reaction was complete (under TLC monitoring) to mono-fluoride kethoxal and its hydrate as a yellow oil (quant.).

### Synthetic Scheme for Phenyl-Kethoxal (3,5-dimethoxyphenylglyoxal)

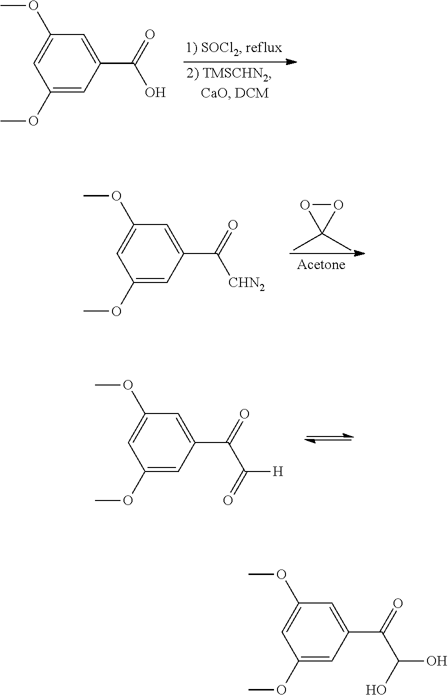

Synthetic route for the phenyl-kethoxal (3,5-dimethoxyphenylglyoxal): 2-diazo-1-(3,5-dimethoxy-phenyl)-ethanone: A mixture of 3,5-dimethoxybenzoic acid (182 mg) and SOCl₂ (1.0 mL) was heated under reflux at 100° C. for 1.5 h. The excess SOCl₂ was removed by vacuum to afford the crude product. The residue was dissolved in anhydrous CH₂C12, cooled to 0° C., and CaO (61 mg) was added. Then, a 2M solution of TMSCHN₂ in diethyl ether (0.5 mL) was added dropwise. The reaction mixture was stirred at 0° C. overnight. The solvent was evaporated and silica gel chromatography (ethyl acetate:petroleum ether=1:10 to 1:3) was performed in order to afford product 2-diazo-1-(3,5-dimethoxy-phenyl)-ethanone as yellow solid (102 mg, 50%).

Phenyl kethoxal or 3,5-dimethoxyphenylglyoxal: According to Adam's procedure, the dimethyldioxirane (DMD) in an acetone solution was prepared. To 2-diazo-1-(3,5-dimethoxy-phenyl)-ethanone (12 mg), DMD-acetone was added, and gas evolution was observed. The reaction mixture was stirred at room temperature until the reaction was complete (under TLC monitoring) to phenyl kethoxal and its hydyate as a yellow oil (quant.).

### Example 2

### Verification of N₃-Kethoxal Reaction with Guanine

The N₃-kethoxal and guanine reaction was verified. Guanine (100 μM, 2 μL), N₃-kethoxal (1 M in DMSO, 1 μL), sodium cacodylate buffer (0.1 M, pH=7.0, 1 μL) and 6 μL ddH₂O were added together into 1.5 mL microcentrifuge tube at 37° C. for 10 min. HRMS C₁₁H₁₄N₈O₄⁺ [M+H]⁺ calculated 323.1216, found 323.1203.

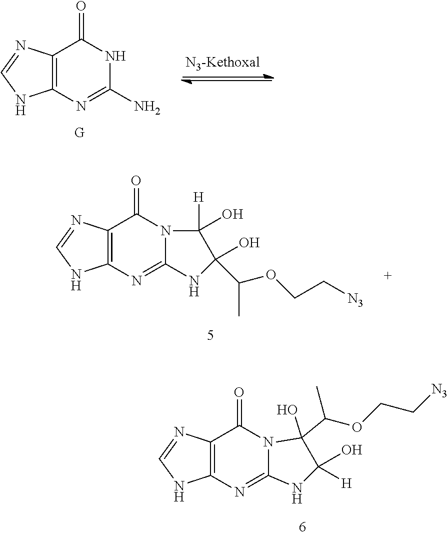 [SMILES: Nc1nc2[nH]cnc2c(=O)[nH]1 ; CC(OCCN=[N+]=[N-])C1(O)Nc2nc3[nH]cnc3c(=O)n2C1O ; CC(OCCN=[N+]=[N-])C1(O)C(O)Nc2nc3[nH]cnc3c(=O)n21]

### Example 3

### The Reaction of N₃-Kethoxal and RNA

The reaction of N₃-kethoxal and RNA was generally performed with the following protocol: 100 pmol RNA oligo and 1 μmol N₃-kethoxal was incubated in total 10 μL solution in PBS buffer at 37° C. for 10 mins. The modified RNA was purified by Micro Bio-Spin™ P-6 Gel Columns (Biorad, 7326222) to remove residual chemicals. The purified labelled RNA can be used for further studies such as mass spectrometry, gel electrophoresis and copper-free click reaction with biotin-DBCO.

Removal N₃-kethoxal modification from N₃-kethoxal labelled RNA. The detailed protocol of N₃-kethoxal modification erasing is described below “N₃-kethoxal-remove sample preparation” in the keth-seq protocol. Generally, the purified N₃-kethoxal modified RNA was incubated with high concentration of GTP (1/2 volume of the reaction solution, final concentration 50 mM) at 37° C. for 6 hours or at 95° C. for 10 mins. Higher temperature benefits the removal the N₃-kethoxal modification.

Fixation of N₃-kethoxal modification in RNA. The labile N₃-kethoxal modification in RNA can be fixed in the presence of borate buffer. The solution of N₃-kethoxal labelled RNA was mixed with 1/10 volume of stock borate buffer (final concentration: 50 mM; stock borate buffer: 500 mM potassium borate, pH 7.0, pH was monitored while adding potassium hydroxide pellets to 500 mM boric acid). The borate buffer fixation was used in various steps of keth-seq protocol, see below.

MALDI-TOF-MS analysis of N₃-kethoxal labelled RNA oligo. The N₃-kethoxal labelled RNA was purified by Micro Bio-Spin™ P-6 Gel Columns. Meanwhile the buffer exchange occurred from PBS buffer to tris buffer that can be directly used in MALDI-TOF-MS experiment without extra desalt step. One microliter of product solution was mixed with one microliter matrix which include 8:1 volume ratio of 2′4′6′-trihydroxyacetophenone (THAP, 10 mg/mL in 50% CH₃CN/H₂O):ammonium citrate (50 mg/mL in H2O). Then the mixture was spotted on the MALDI sample plate, dried and analyzed by Bruker Ultraflextreme MALDI-TOF-TOF Mass Spectrometers.

### Example 4

### Phenol-Kethoxal and Diphenol-Kethoxal

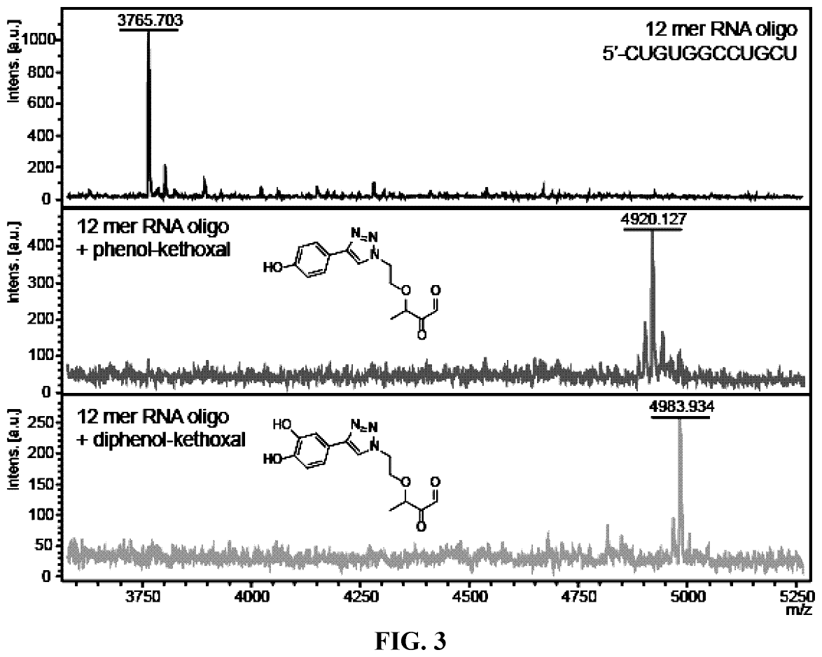

To test the labeling activity of phenol-kethoxal and diphenol-kethoxal, the two compounds were incubated with a 12-mer synthetic RNA oligo containing four guanine bases, respectively. After 10 min, the reactions were cleaned-up and analyzed by MALDI-TOF. Both phenol-kethoxal and diphenol-kethoxal label the oligo efficiently, with all four guanines on all oligo molecules modified, see FIG. 3.

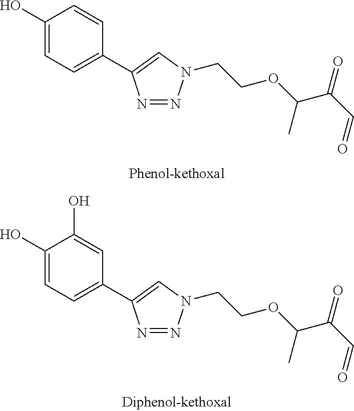 [SMILES: CC(OCCn1cc(-c2ccc(O)cc2)nn1)C(=O)C=O ; CC(OCCn1cc(-c2ccc(O)c(O)c2)nn1)C(=O)C=O]

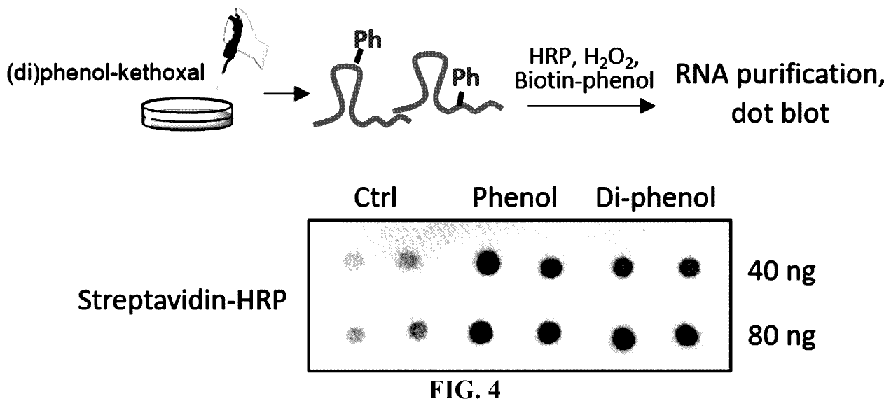

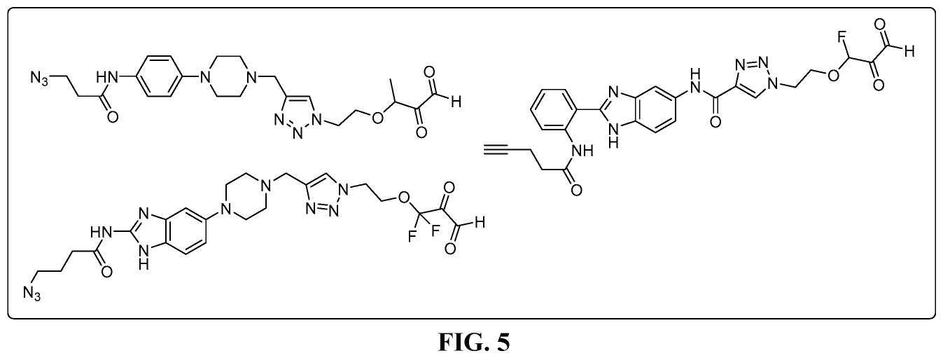

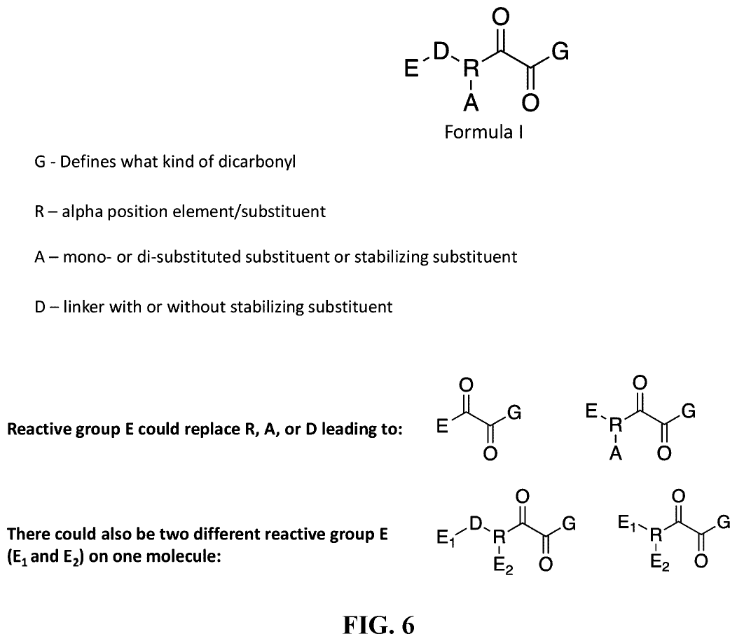

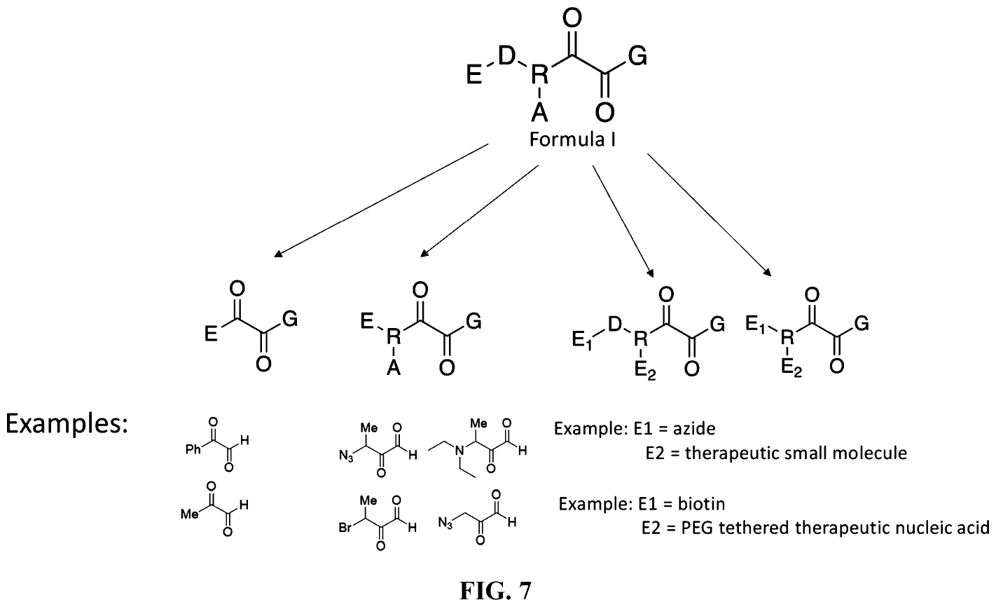

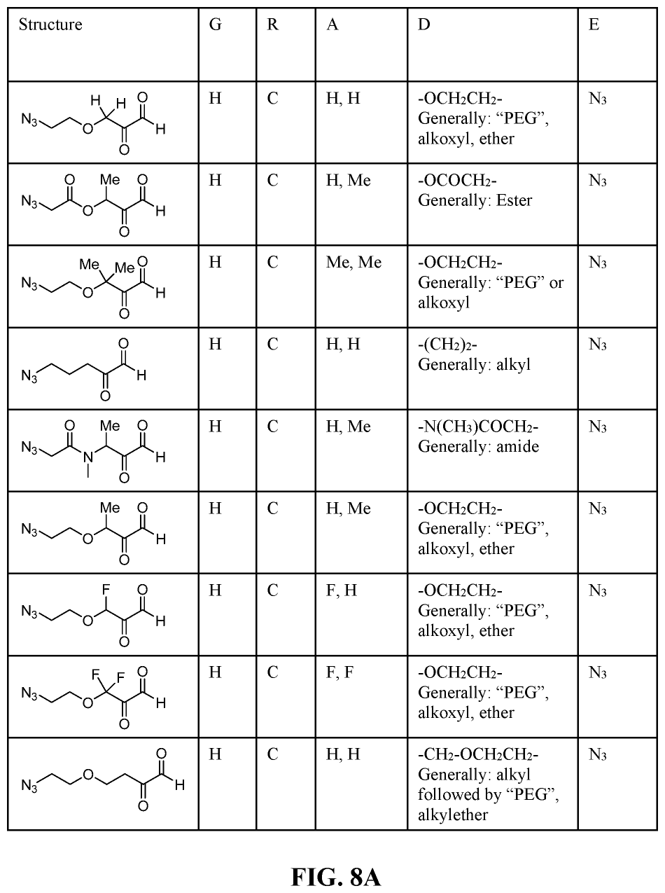

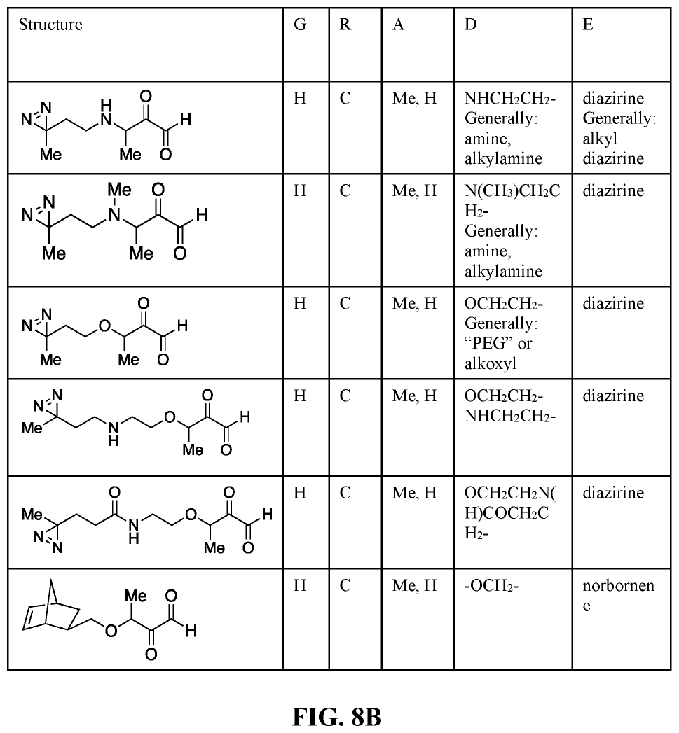

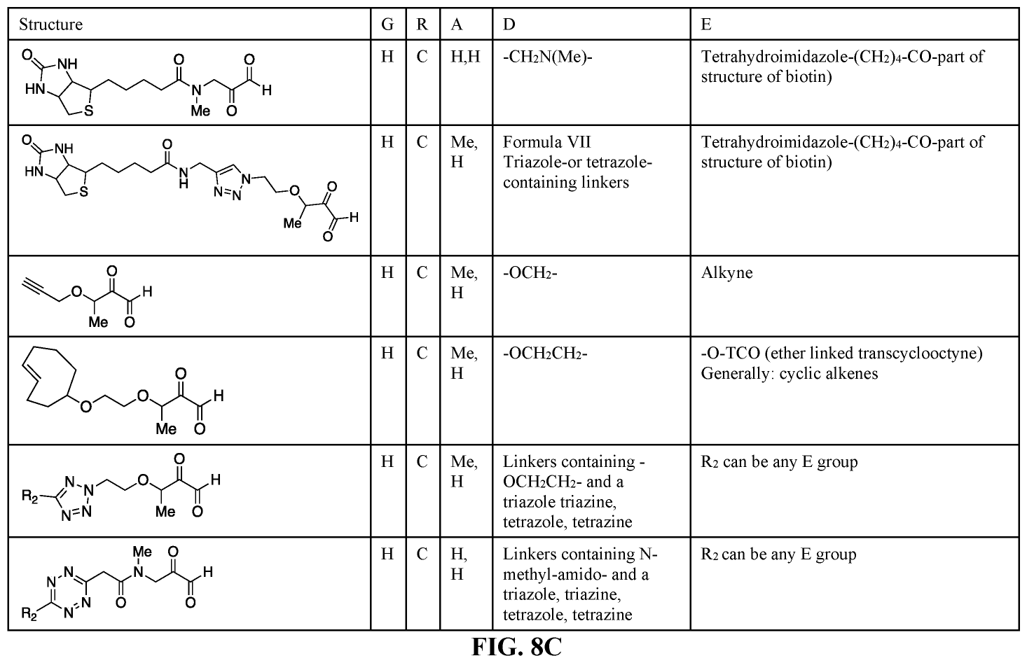

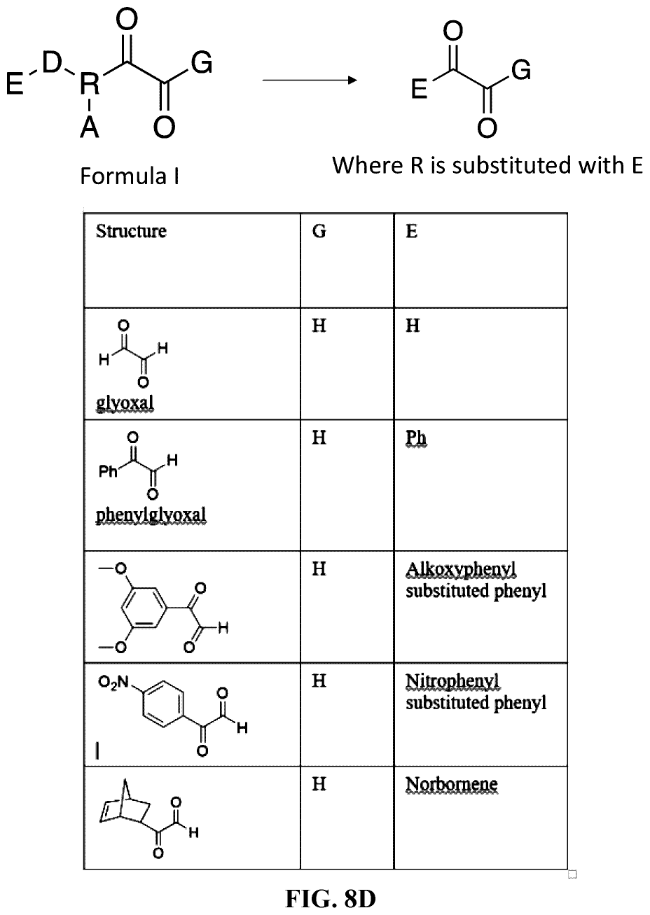

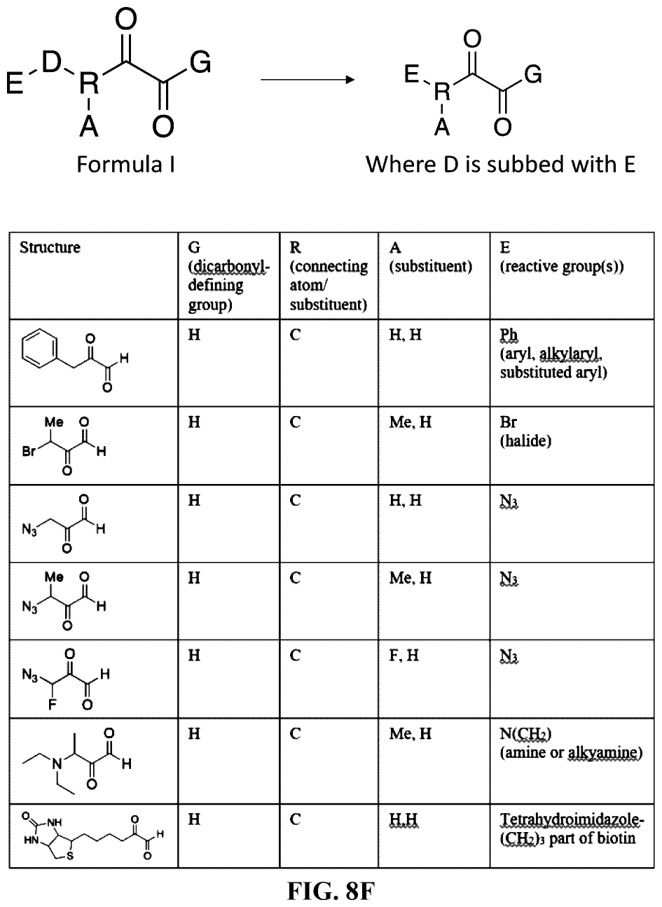

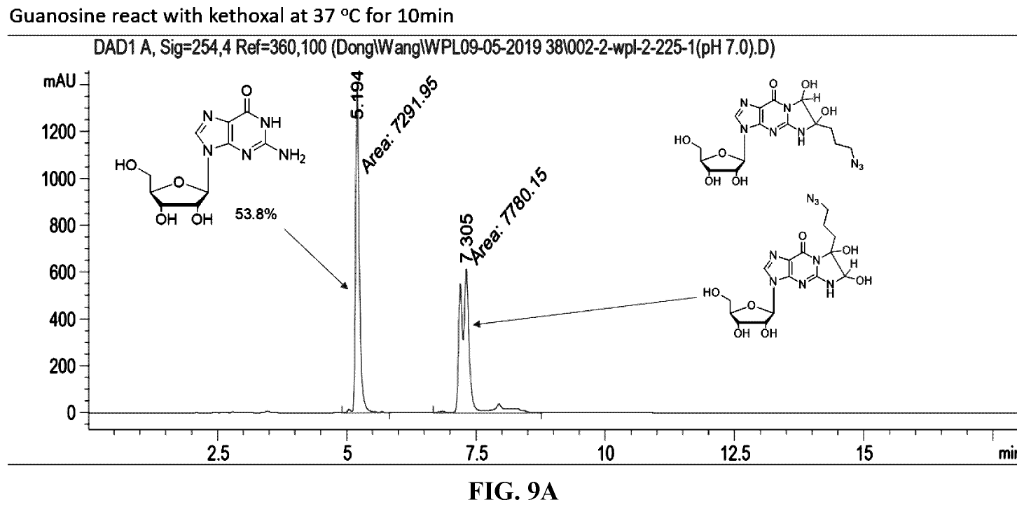

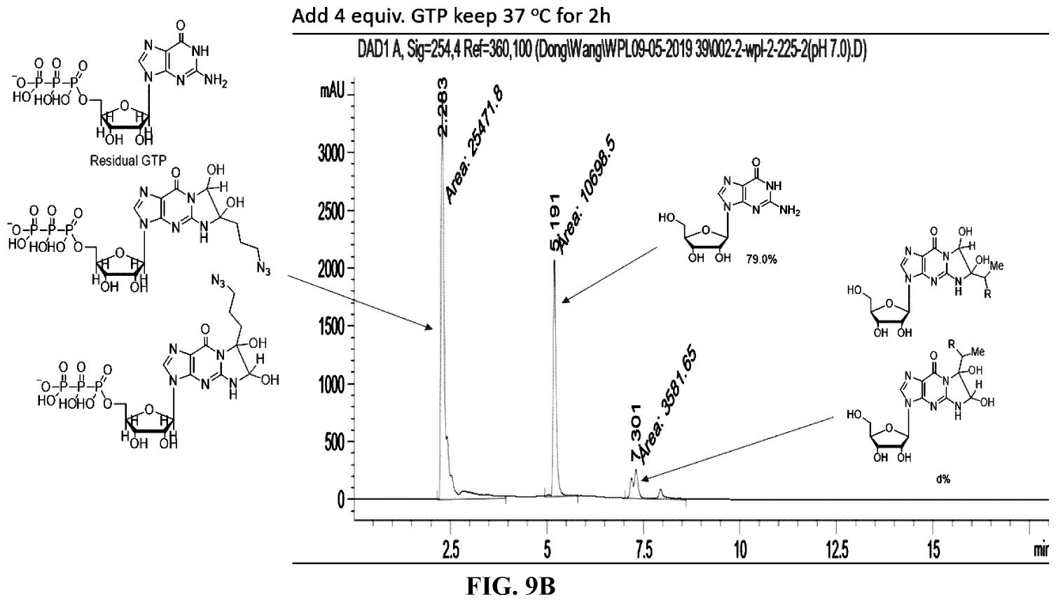

A second set of test were performed to test cell permeability of phenol-kethoxal and diphenol-kethoxal and if the labeling enhances radical-mediated biotinylation. Cells were treated with phenol-kethoxal and diphenol-kethoxal for 10 min, respectively, and RNA isolated from treated cells. An in vitro biotinylation reaction was performed by mixing these kethoxal derivative-labeled RNAs with biotin-phenol, horseradish peroxidase (HRP), and H₂O₂, see FIG. 4. HRP is an enzyme that mimics APEX with higher radical generation activity in vitro. The biotinylated RNAs were purified and subjected to dot blot analysis. Both phenol-kethoxal-modified and diphenol-kethoxal-modified RNAs show stronger biotin signals compared with the control sample, suggesting (di)phenol-kethoxal could enhance radical-mediated biotinylation and show potentials for high-efficiency APEX-mediated proximity labeling in live cells.

### Example 5

### Experiment Procedure for Single-Stranded DNA (SSDNA) Mapping

ssDNA is performed by: (1) Prepare labeling medium by adding 5 μL pure a kethoxal derivative (e.g., N₃-kethoxal) to 5 mL pre-warmed cell culture medium for each 10 cm dish. (2) Incubate cells in the labeling medium for 10 min at 37° C., 5% CO₂. (3) For transcription inhibition experiments, cells were treated for 2 h under 100 μM DRB or 1 μM triptolide before incubated in kethoxal-derivative containing medium. (4) Harvest cells after the 10 min incubation, isolate total DNA from cells by PureLink genomic DNA mini kit according to the manufacturer's protocol. (5) Suspend 5 μg total DNA in 85 μL H2O, then add 10 μL 10×PBS and 5 μL 20 mM DBCO-PEG4-biotin (DMSO solution), incubate the mixture at 37° C. for 2 h. (6) Add 5 μL RNase A to the reaction mixture, incubate the mixture at 37° C. for another 15 min. (7) Recover DNA from the reaction mixture by DNA Clean & Concentrator kit according to the manufacturer's protocol.

Libraries were constructed by different commercial library construction kits with similar results obtained. Two examples include:

(8a) The use of Accel-NGS Methyl-seq DNA library kit (Swift): (i) Fragment 2 μg of recovered DNA from step 7 by sonication under 30 s-on/30 s-off setting for 30 cycles (ii) Save 5% of the fragmented DNA for input, use the rest 95% to enrich biotin-tagged DNA by 10 μL pre-washed Streptavidin Cl beads according to the manufacturer's protocol with minor changes. Beads were washed 3 times in 1× binding and wash buffer with 0.05% tween-20 before re-suspended in 95 μL 2× binding and wash buffer with 0.1% tween-20. Beads were mixed with DNA and the mixture was incubated at room temperature for 15 min with gentle rotation. After incubation, beads were washed 5 times with 1× binding and wash buffer with 0.05% tween-20 (iii) Elute the enriched DNA by heating the beads in 30 μL H₂O at 95° C. for 10 min. Treat the saved input at 95° C. for 10 min at the same time. The put both input and IP samples on ice immediately (iv) Proceed to library construction according the protocol from the Accel-NGS Methyl-seq DNA library kit.

(8b) The use of Kapa Hyper Plus kit (Kapa Biosystems): (i) Suspend 1 μg total DNA in 35 μL H₂O, add 5 μL Kapa fragmentation buffer and 10 μL Kapa fragmentation enzyme. Incubate the mixture at 37° C. for 30 min. (ii) Recovery fragmented DNA by DNA Clean & Concentrator kit according to the manufacturer's protocol (iii) Perform A-tailing and adapter ligation according the protocol from Kapa Hyper Plus kit. (iv) Save 5% of the DNA for input, use the rest 95% to enrich biotin-tagged DNA by 10 μL pre-washed Streptavidin Cl beads according to the manufacturer's protocol with minor changes. Beads were washed 3 times in 1× binding and wash buffer with 0.05% tween-20, before re-suspended in 95 μL 2× binding and wash buffer with 0.1% tween-20. Beads were mixed with DNA and the mixture was incubated at room temperature for 15 min with gentle rotate. After incubation, beads were washed 5 times with 1× binding and wash buffer with 0.05% tween-20 (v) Elute the enriched DNA by heating the beads in 25 μL H₂O at 95° C. for 10 min. (vi) PCR amplify the libraries for both input and IP samples according to the protocol from Kapa Hyper Plus kit. (9) Sequence libraries on Nextseq SR80 mode and perform downstream analysis.

### Example 6

### Experiment Procedure for Kethoxal-Assisted RNA-RNA Interaction (KARRI)

KRRI is performed by: (1) Suspend live cells in 1% formaldehyde solution at 1×10⁶/mL and incubate at room temperature for 10 min with gentle rotate. Then quench this reaction by adding glycine to a final concentration of 125 mM and rotate the mixture at room temperature for 5 min. For translation inhibitor treatment, cells were treated with 100 μg/mL cycloheximide or 3 μg/mL harringtonine at 37° C. for 10 min. (2) Collect and take 2×10⁶ cells. Dilute Kethoxal derivative (e.g., N₃-kethoxal) by 1:5 using DMSO. Make a labeling buffer by adding 10 μL Kethoxal derivative into 290 μL lysis buffer (10 mM Tris-HCl pH 8.0, 10 mM NaCl, 0.2 IGEPAL CA630) with 3 μL 100× proteinase inhibitor cocktail. (3) Suspend cells in labeling buffer and rotate at room temperature for 30 min, then centrifuge at 2500 g for 5 min at 4° C. to collect cells. (4) Wash cell pellets with 500 μL ice-cold lysis buffer for 3 times. (5) Suspend the pellet in 500 μL MeOH containing 10 mM dendrimers, rotate for 1 h at 37° C. Then centrifuge at 2500 g for 5 min at 4° C. to collect cells. (6) Wash cell pellet twice with 500 μL ice-cold lysis buffer. (7) Resuspend cells in 385 μL lysis buffer, add 50 μL 10% SDS, 30 μL proteinase K, 10 μL RNase inhibitor, 25 μL 500 mM K3B03, shake at 65° C. for 2 h. (8) Add 500 μL phenol-chloroform to extract RNA and purify RNA by EtOH precipitation. (9) Suspend RNA pellets in 104 μL H2O, add 12 μL 10×DNase I buffer (100 mM Tris-HCl pH 7.4, 25 mM MgCl₂, 1 mM CaCl₂), 2 μL DNase I (Thermo), 2 μL RNase inhibitor, and incubate at 37° C. for 30 min with gentle shaking. (10) Add 130 μL 2× proteinase K buffer (100 mM Tris-HCl pH 7.5, 200 mM NaCl, 2 mM EDTA, 1% SDS), 10 μL proteinase K to the reaction, incubate at 65° C. for 30 min with shaking (11) Extract RNA with 300 μL phenol-chloroform and purify RNA by EtOH precipitation. (12) Suspend RNA pellets in 61 μL H2O, add 7 μL 10× fragmentation buffer (Thermo), 2 μL RNase inhibitor, incubate at 70° C. for 15 min, then add 8 μL fragmentation stop buffer (Thermo) and put the sample on ice immediately to quench the reaction. (13) Enrich crosslinked RNA by using 30 μL pre-washed Streptavidin Cl beads according to the manufacturer's protocol with minor changes. Beads were washed 3 times in 1× binding and wash buffer with 0.05% tween-20, before re-suspended in 80 μL 2× binding and wash buffer with 0.1% tween-20. Beads were mixed with DNA and the mixture was incubated at room temperature for 30 min with gentle rotate. After incubation, beads were washed 3 times with 1× binding and wash buffer with 0.05% tween-20 and once with 1×PNK buffer (NEB). (14) Suspend beads in 41 μL H2O, 5 μL 10×PNK buffer (NEB), 3 μL T4 PNK (NEB), 1 μL RNase inhibitor and shake at 37° C. for 30 min, then add another 3 μL T4 PNK and 6 μL 10 mM ATP, shake at 37° C. for another 30 min. (15) Wash beads twice with 1× binding and wash buffer with 0.05% tween-20, once with 1× ligation buffer (NEB). (16) Suspend beads in 668 μL H2O, 100 μL 10× ligase buffer (NEB), 10 μL RNase inhibitor, 2 μL 10 mM ATP, 20 μL T4 RNA ligase 2 (high concentration) (NEB), 200 μL 50% PEG 8000, rotate at 16° C. for 16 h. (17) Wash beads twice with 1× binding and wash buffer with 0.05% tween-20, once with H2O. Then elute RNA by heating the beads in 30 μL H₂O and shaking beads at 95° C. for 10 min. (18) Take half of the recovered RNA for library construction using the SMARTer Stranded Total RNA-seq Kit v2-Pico Input (Takara) by following the protocol from the manufacturer. (19) Sequence libraries on Novaseq PE150 mode and perform downstream analysis.

### Example 7

### Activity of Representative Kethoxal Derivatives

Reactivity and reversibility modulation of kethoxal derivatives. The reactivity and the reversibility of kethoxal derivatives can be tuned by adding a series of functional groups onto the glyoxal moiety. Here we studied the effect of reaction pH, electron donating/withdrawing groups, and steric on the reactivity and reversibility of kethoxal derivatives. We observed that the reactivity and reversibility are pH-dependent. Hydrogen bond acceptors at the α-position of the ketone largely enhance the reactivity by stabilizing the formed adduct through H-bonding with the guanosine amine proton. While most tested kethoxal derivatives show reversibility with GTP as competitor, less reactive molecules are generally more reversible. These studies deeper our understanding about the chemical properties of these molecules and therefore, provide theoretical structure-activity guidance and validates the feasibility of applying these molecules to both genomic studies (such as ssDNA and RNA labelling applications) and kethoxal-based therapeutic purposes.

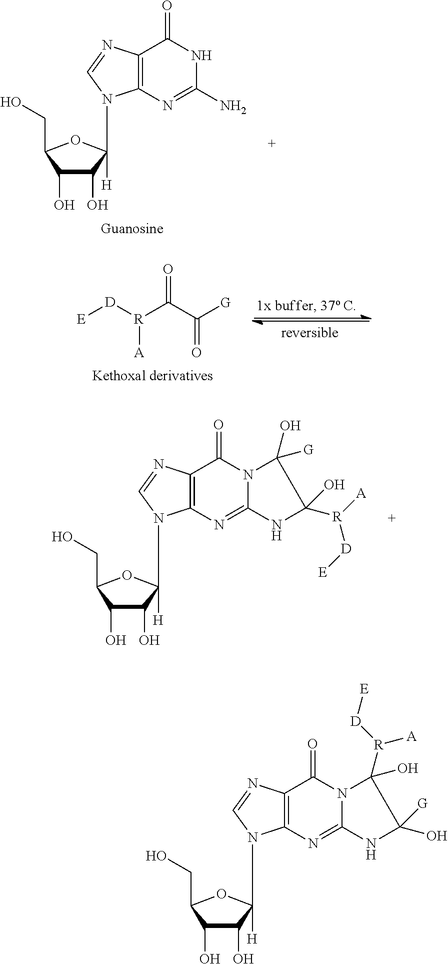

1. Kethoxal derivatives are more reactive with guanosine at basic conditions. Conversion rates of guanosine at different pH conditions are shown in Table 1. Shown below is an example with a phenyl-substituted kethoxal derivative. In the image of the reaction below, guanosine is depicted as S1 and the kethoxal derivative is depicted as S2.

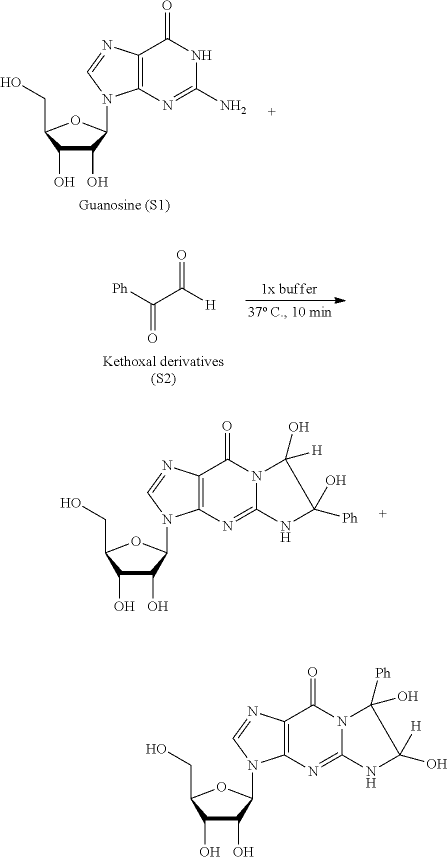

TABLE 1
The effect of pH on reactivity.

| | S1:S2 = 1:1 | S1:S2 = 1:2 | S1:S2 = 1:3 | S1:S2 = 1:5 |
|---|---|---|---|---|
| pH = 7.0 | 18.8% | 37.6% | 51.0% | 67.0% |
| pH = 7.8 | 32.2% | 51.2% | 66.2% | 80.1% |

2. Electronic and steric effects can modulate the reactivity of kethoxal derivatives. Conversion rates of guanosine with different kethoxal derivatives at pH 7.8 are shown in Tables 2A and 2B. In the image of the reaction below, guanosine is depicted as S1 and the kethoxal derivatives are depicted as S2.

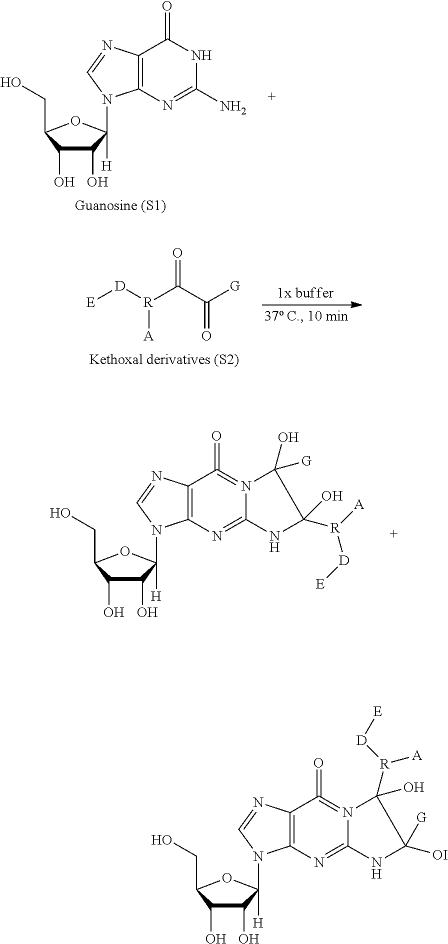

TABLE 2A
Reactivity of different kethoxal derivatives at pH = 7.8.

| | S1:S2 = 2:1 | S1:S2 = 1:1 | S1:S2 = 1:2 | S1:S2 = 1:3 | S1:S2 = 1:5 | S1:S2 = 1:10 |
|---|---|---|---|---|---|---|
| 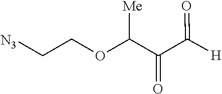 [SMILES: CC(OCCN=[N+]=[N-])C(=O)C=O] | 51.6% | 86.9% | 97.4% | | | |
| 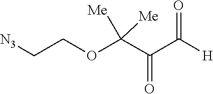 [SMILES: CC(C)(OCCN=[N+]=[N-])C(=O)C=O] | 51.3% | 81.6% | 97.4% | | | |
| 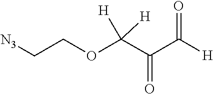 [SMILES: [N-]=[N+]=NCCOCC(=O)C=O] | 51.3% | 78.6% | 95.4% | | | |
| 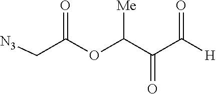 [SMILES: CC(OC(=O)CN=[N+]=[N-])C(=O)C=O] | 43.6% | 77.5% | 92.1% | | | |
| 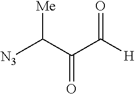 | 38.0% | 71.2% | 90.3% | 96.5% | | |
| 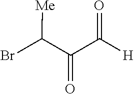 | 35.8% | 67.2% | 89.9% | 92.2% | | |
| 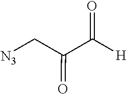 | 33.4% | 60.4% | 79.4% | 85.4% | | |

TABLE 2B
Reactivity of different kethoxal derivatives at pH = 7.8 (continued)

| | S1:S2 = 2:1 | S1:S2 = 1:1 | S1:S2 = 1:2 | S1:S2 = 1:3 | S1:S2 = 1:5 | S1:S2 = 1:10 |
|---|---|---|---|---|---|---|
| 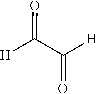 | 32.1% | 49.8% | 67.3% | 89.7% | 98.3% | 98.3% |
| 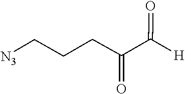 | 23.8% | 48.0% | 70.5% | 88.9% | 89.2% | |
| 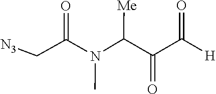 | | 40.2% | 66.7% | 74.9% | 83.2% | |
| 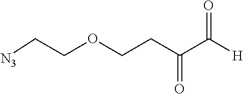 [SMILES: [N-]=[N+]=NCCOCCC(=O)C=O] | 25.2% | 41.1% | 60.0% | 66.4% | 73.6% | |
| 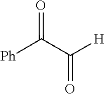 | | 32.2% | 51.2% | 66.2% | 80.1% | |
| 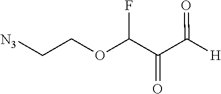 [SMILES: [N-]=[N+]=NCCOC(F)C(=O)C=O] | | 30.9% | 49.6% | 69.5% | 76.7% | 81.6% |
| 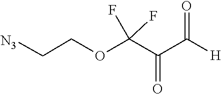 [SMILES: [N-]=[N+]=NCCOC(F)(F)C(=O)C=O] | 8.5% | 14.7% | 28.9% | 38.7% | 63.1% | |

3. Reaction pH has different effects on kethoxal reactivity depending on substituents on the kethoxal derivatives. Conversion rates of guanosine with different kethoxal derivatives at pH 7.0 are shown in Tables 3A and 3B.

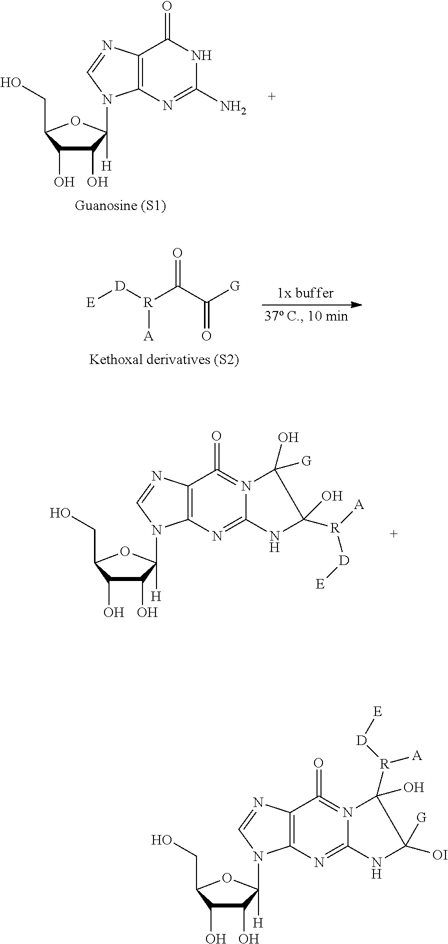

TABLE 3A
Reactivity of different kethoxal derivatives at pH = 7.0.

| | S1:S2 = 2:1 | S1:S2 = 1:1 | S1:S2 = 1:2 | S1:S2 = 1:4 | S1:S2 = 1:10 |
|---|---|---|---|---|---|
| 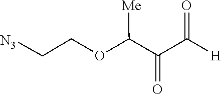 [SMILES: CC(OCCN=[N+]=[N-])C(=O)C=O] | 39.6% | 70.6% | 93.7% | | |
| 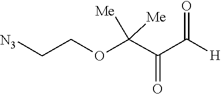 [SMILES: CC(C)(OCCN=[N+]=[N-])C(=O)C=O] | 23.6% | 46.7% | 76.6% | | |
| 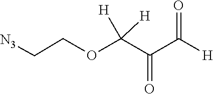 [SMILES: [N-]=[N+]=NCCOCC(=O)C=O] | 30.3% | 52.2% | 79.2% | | |
| 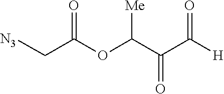 [SMILES: CC(OC(=O)CN=[N+]=[N-])C(=O)C=O] | 29.5% | 50.1% | 81.3% | | |
| 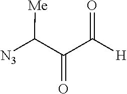 | 22.0% | 46.7% | 79.2% | 95.3% | |
| 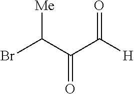 | 22.4% | 40.4% | 81.2% | | |
| 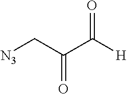 | 16.8% | 33.7% | 55.4% | 76.3% | |

TABLE 3B
Reactivity of different kethoxal derivatives at pH = 7.0 (continued)

| | S1:S2 = 2:1 | S1:S2 = 1:1 | S1:S2 = 1:2 | S1:S2 = 1:4 | S1:S2 = 1:0 |
|---|---|---|---|---|---|
| 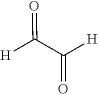 | 7.5% | 17.0% | 30.0% | 59.7% | |
| 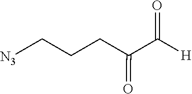 | 19.8% | 40.8% | 63.2% | 87.2% | |
| 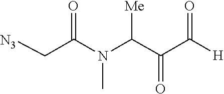 | 20.4% | 46.2% | 64.2% | 84.7% | |
| 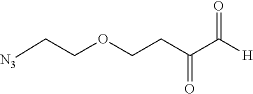 [SMILES: [N-]=[N+]=NCCOCCC(=O)C=O] | 16.8% | 38.3% | 49.6% | | |
| 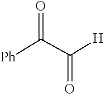 | 9.9% | 22.5% | 33.2% | 51.5% | |
| 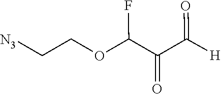 [SMILES: [N-]=[N+]=NCCOC(F)C(=O)C=O] | 3.2% | 6.4% | 8.2% | 16.5% | 24.7% |
| 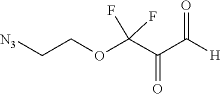 [SMILES: [N-]=[N+]=NCCOC(F)(F)C(=O)C=O] | 0 | 1.4% | 1.9% | 3.8% | 9.8% |
| 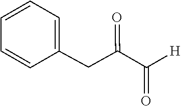 [SMILES: O=CC(=O)Cc1ccccc1] | 9.0% | 22.5% | 30.4% | | |

4. Improving product stability with hydrogen bonding. When guanosine reacts with kethoxal derivatives, a proton on the guanosine amine is capable of engaging in hydrogen bond formation. Therefore, kethoxal derivatives with H-bond-accepting substituents stabilize the product formed and facilitate the reaction. Conversely, derivatives without H-bonding substituents may be relatively less reactive. Shown in the image is N₃-kethoxal, which has a ether-containing D linker (based on Formula I); this H-bond accepting moiety stabilizes the product.

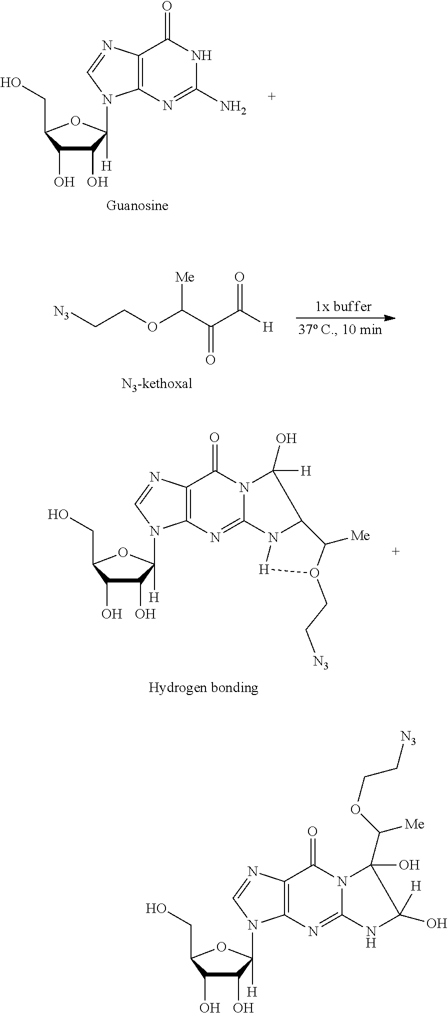

5. Testing the reversibility of kethoxal derivatives by adjusting pH. As the reactivity of most kethoxal derivatives is higher under basic conditions, we first applied a high pH (pH=10.1) to transform kethoxal derivatives into the kethoxal-guanosine adduct. We then adjusted the pH to 5.8 and measured extent of product dissociation. Kethoxal derivatives and guanosine were mixed at 1:1 ratio. Results are shown in Table 4 (the numbers show the conversion of guanosine).

TABLE 4
The reversibility of kethoxal derivatives

| | pH = | pH = | pH = | pH = |
|---|---|---|---|---|
| | 10.1, | 5.8, | 5.8, | 5.8, |
| | 10 min | 10 min | 4 h | 24 h |
| 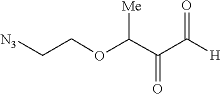 [SMILES: CC(OCCN=[N+]=[N-])C(=O)C=O] | 79.8% | 79.8% | 80.2% | 81.8% |
| 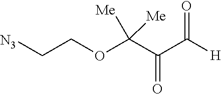 [SMILES: CC(C)(OCCN=[N+]=[N-])C(=O)C=O] | 77.0% | | 77.6% | 80.3% |
| 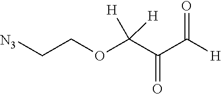 [SMILES: [N-]=[N+]=NCCOCC(=O)C=O] | 74.6% | | 75.0% | 76.1% |
| 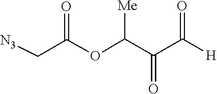 [SMILES: CC(OC(=O)CN=[N+]=[N-])C(=O)C=O] | 75.5% | | 77.3% | 77.2% |
| 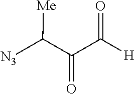 | 65.9% | 65.6% | 65.2% | 58.7% |
| 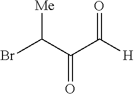 | 62.7% | | 64.3% | 62.9% |
| 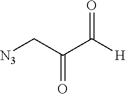 | 24.5% | 23.8% | 21.6% | 20.8% |
| 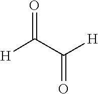 | 84.7% | 85.2% | 84.4% | 84.5% |
| 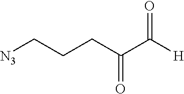 | 30.2% | | 19.0% | 14.7% |
| 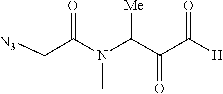 | 35.6% | | 31.9% | 26.5% |
| 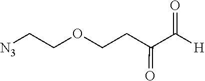 [SMILES: [N-]=[N+]=NCCOCCC(=O)C=O] | 19.7% | | 16.6% | |
| 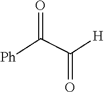 | 28.3% | 12.2% | 10.7% | 12.7% |
| 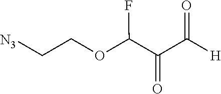 [SMILES: [N-]=[N+]=NCCOC(F)C(=O)C=O] | 46.2% | 50.1% | 57.1% | 58.2% |
| 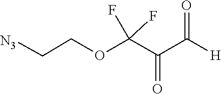 [SMILES: [N-]=[N+]=NCCOC(F)(F)C(=O)C=O] | 41.5% | 49.2% | 55.1% | 54.7% |

6. Testing the reversibility of kethoxal derivatives by using GTP for competition. We first mixed kethoxal derivatives and guanosine to form guanosine-kethoxal adducts. Kethoxal derivatives and guanosine were mixed at a 1:1 ratio. After 10 min, we added excess guanosine 5′-triphosphate (GTP), to as a competitor. Excess GTP is expected to competitively react with the kethoxal derivative, resulting in increased free guanosine. This free guanosine is detected by LCMS and used to determine relative reversibility afforded by the substituents on the kethoxal derivative (see reaction image and LCMS images).

Results are shown in Table 5 (the numbers show the conversion of guanosine) and an example LCMS image is shown below.

The kethoxal derivative reacts with guanosine to form the kethoxal-guanosine adduct.

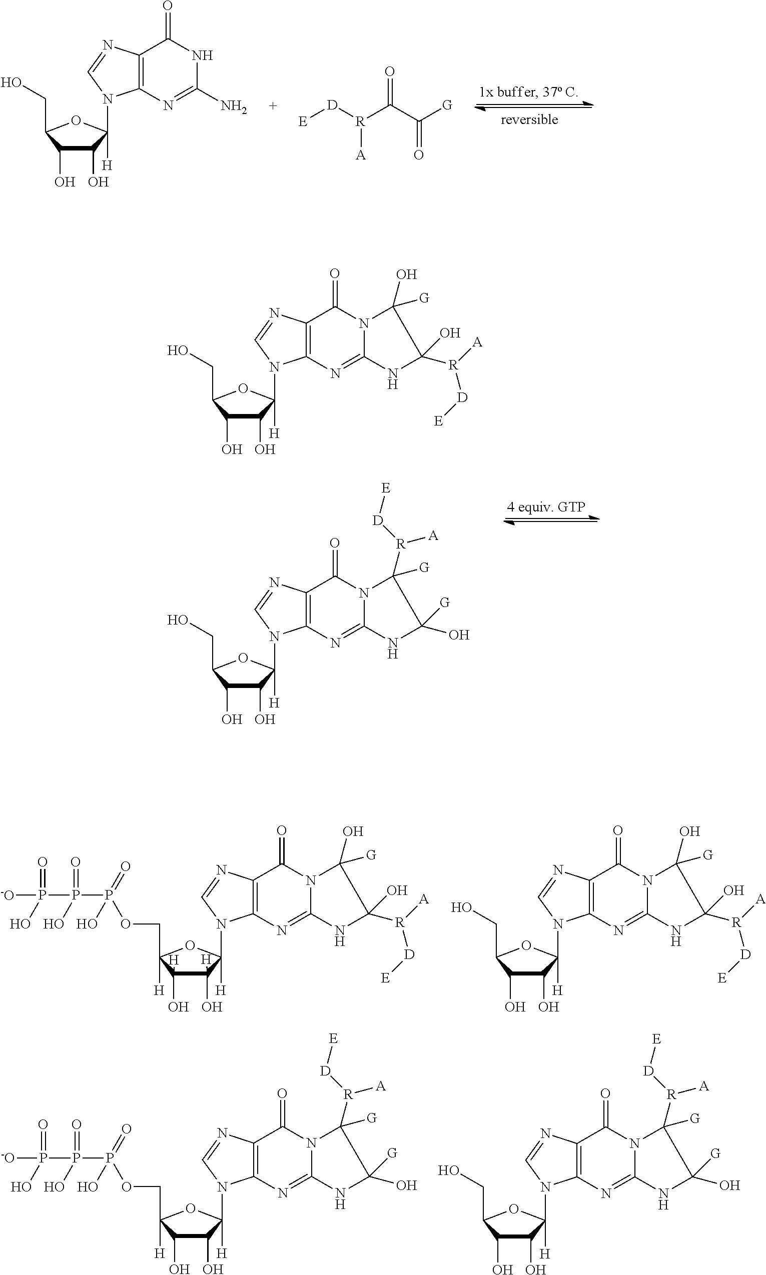

TABLE 5
The reversibility of kethoxal derivatives under competition condition

| | pH = 7.0, | pH = 7.0, | pH = 7.0, |
|---|---|---|---|
| | 10 min | 2 h | 24 h |
| 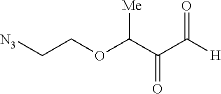 [SMILES: CC(OCCN=[N+]=[N-])C(=O)C=O] | 71.4% | 60.8% | 28.9% |
| 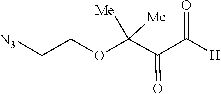 [SMILES: CC(C)(OCCN=[N+]=[N-])C(=O)C=O] | 51.6% | 55.9% | 33.6% |
| 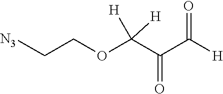 [SMILES: [N-]=[N+]=NCCOCC(=O)C=O] | 47.4% | 29.7% | 27.4% |
| 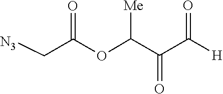 [SMILES: CC(OC(=O)CN=[N+]=[N-])C(=O)C=O] | 54.4% | 44.6% | 37.5% |
| 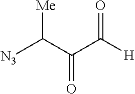 | 56.5% | 40.9% | |
| 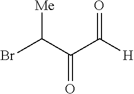 | 46.2% | 38.2% | 18.6% |
| 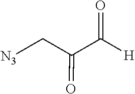 | 34.6% | 24.8% | 12.4% |
| 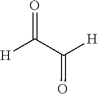 | 52.1% (pH = 7.8) | 64.3% (pH = 7.8) | 30.7% (pH = 7.8) |
| 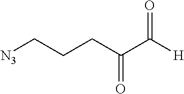 | 46.2% | 21.0% | 22.1% |
| 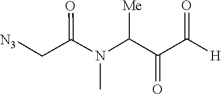 | 41.8% | 26.1% | 23.4% |
| 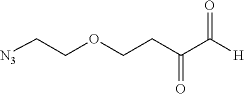 [SMILES: [N-]=[N+]=NCCOCCC(=O)C=O] | 41.3% | 12.6% | 11.2% |
| 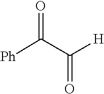 | 25.7% | 12.3% | 4.4% |
| 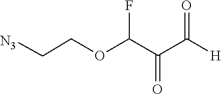 [SMILES: [N-]=[N+]=NCCOC(F)C(=O)C=O] | 6.4% | 18.6% | 22.8% |
| 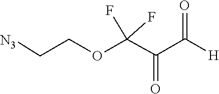 [SMILES: [N-]=[N+]=NCCOC(F)(F)C(=O)C=O] | 51.2% (pH = 10.1) | 42.4% (pH = 10.1) | 22.2% (pH = 10.1) |
| 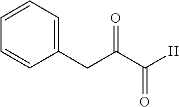 [SMILES: O=CC(=O)Cc1ccccc1] | 21.8% | 9.6% | 8.4% |
| 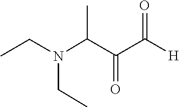 | 66.9% | 66.1% | 36.3% |
| 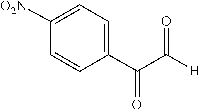 [SMILES: O=CC(=O)c1ccc([N+](=O)[O-])cc1] | 48.0% | 13.5% | |

1. A kethoxal complex comprising an agent coupled to a kethoxal derivative having a general formula of Formula I: 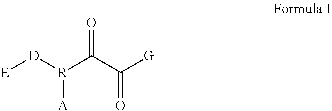
wherein E is a reactive functional group selected from alkynes, azides, strained alkynes, dienes, dieneophiles, alkoxyamines, carbonyls, phosphines,
hydrazides, thiols, and alkenes;
D is optionally a linker or a direct bond;
R is a connecting group;
A one or two substituents selected from H, F, CF₃, CF₂H, CFH₂, CH₃, alkyl group, or combinations thereof, or A is a second E moiety selected independent of the first E moiety; and
G is H, F, CF₃, CF₂H, CFH₂, CH₃, or an alkyl group.

2. The kethoxal complex of claim 1, wherein E is selected from a substituted alkyl, heteroalkyl, substituted heteroalkyl, heteroaryl, or substituted heteroalkyl. In some aspects, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene.

3. The kethoxal complex of claim 1 or 2, wherein D is a linker selected from one or more of an ester, amide, tetrazine, tetrazole, triazine, triazole, aryl groups, heterocycle, sulfonamide, a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. D can be —N(CH₃)—, —OCH₂—, —N(CH₃)COCH₂—, or

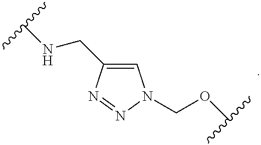 [SMILES: *NCc1cn(CO*)nn1]

4. The kethoxal complex of claim 3, wherein the linker is a concatamer of 1, 2, 3, 4, 5, 6, 7, 8, 9, 10, or more of the linkers.

5. The kethoxal complex of any one of claims 1 to 3, wherein R is selected from a substituted or unsubstituted carbon, nitrogen, aryl, alkylaryl, or heterocycle.

6. The kethoxal complex of any one of claims 1 to 5, wherein G is H; R is C; A is CH₃; D is —OCH₂CH₂-triazole-pyridine-aryl-amide-CH₂CH₂, and E is N₃ (azide); (ii) G is H; R is C, A is F, D is —OCH₂CH₂-triazole-amide-benzoimidazole-phenyl-NHCO—CH₂CH₂, and E is alkyne; (iii) G is H, R is C, A is a di-fluoro substituent of R, D is —OCH₂CH₂-triazole-CH₂-pyridine-benzoimidazole-NHCO—CH₂CH₂CH₂—, and E is N₃ (azide); (iv) G is H, R is C, A is methyl, D is —OCH₂CH₂-triazole-, and E is phenol or diphenol.

7. The kethoxal complex of claim 1, wherein the kethoxal complex is selected from 3-azido-2-oxopropanal, 3-azido-2-oxobutanal, 3-azido-3-fluoro-2-oxopropanal, 2-oxo-6-(2-oxohexahydro-1H-thieno[3,4-d]imidazol-4-yl)hexanal, 2-((1S,4S)-bicyclo[2.2.1]hept-5-en-2-yl)-2-oxoacetaldehyde, 2-oxo-2-phenylacetaldehyde, 2-(3,5-dimethoxyphenyl)-2-oxoacetaldehyde, 2-(4-nitrophenyl)-2-oxoacetaldehyde, N-(2,3-dioxopropyl)-N-methyl-5-(2-oxohexahydro-1H-thieno[3,4-d]imidazol-4-yl)pentanamide, N-((1-(2-((3,4-dioxobutan-2-yl)oxy)ethyl)-1H-1,2,3-triazol-4-yl)methyl)-5-(2-oxohexahydro-1H-thieno[3,4-d]imidazol-4-yl)pentanamide, 2-oxo-3-(prop-2-yn-1-yloxy)butanal, (E)-3-(2-(cyclooct-4-en-1-ylamino)ethoxy)-2-oxobutanal, 3-(2-azidoethoxy)-2-oxopropanal, 3,4-dioxobutan-2-yl 2-azidoacetate, 3-(2-azidoethoxy)-3-methyl-2-oxobutanal, 5-azido-2-oxopentanal, 2-azido-N-(3,4-dioxobutan-2-yl)-N-methylacetamide, 3-(2-azidoethoxy)-2-oxobutanal, 3-(2-azidoethoxy)-3-fluoro-2-oxopropanal, 3-(2-azidoethoxy)-3,3-difluoro-2-oxopropanal, 4-(2-azidoethoxy)-2-oxobutanal, or 3-(((1S,4S)-bicyclo[2.2.1]hept-5-en-2-yl)methoxy)-2-oxobutanal.

8. A kethoxal complex comprising an agent coupled to a kethoxal derivative having a general formula of Formula III: 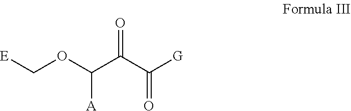
wherein E is a click chemistry moiety selected from alkynes, azides, strained alkynes, dienes, dieneophiles, alkoxyamines, carbonyls, phosphines, hydrazides, thiols, and alkenes; and A and G are independently selected from H, CF₃, CF₂H, CFH₂, or CH₃.

9. A kethoxal complex comprising an agent coupled to a kethoxal derivative having a general formula of Formula IV: 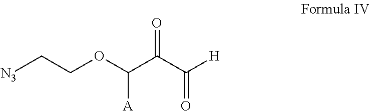
wherein A is a substituent selected from H, F, CF₃, CF₂H, CFH₂, or CH₃ or is a linker.

10. A kethoxal complex comprising an agent coupled to a kethoxal derivative having the formula: 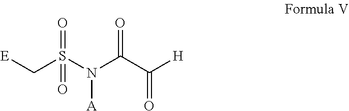
wherein E is a click chemistry moiety selected from alkynes, azides, strained alkynes, dienes, dieneophiles, alkoxyamines, carbonyls, phosphines, hydrazides, thiols, and alkenes; and A is independently selected from H, F, CF₃, CF₂H, CFH₂, or CH₃.

11. A kethoxal complex comprising an agent coupled to a kethoxal derivative having the formula: 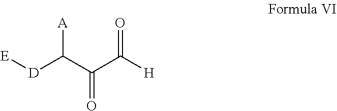
wherein A is hydrogen or methyl; D is a linker; and E is reactive functional group.

12. The kethoxal complex of claim 11, wherein D is a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R⁶ is alkyl, substituted alkyl, cycloalkyl, substituted cycloalkyl, heteroalkyl, substituted heteroalkyl, aryl, substituted aryl, heteroaryl, or substituted heteroarylaryl.

13. The kethoxal complex of claim 11, wherein D is substituted with a reactive group.

14. The kethoxal complex of claim 13, wherein the reactive group is a click chemistry moiety.

15. The kethoxal complex of claim 11, wherein D is —N(CH₃)—, —OCH₂—, —N(CH₃)COCH₂—, or a group having the chemical formula of Formula VII,

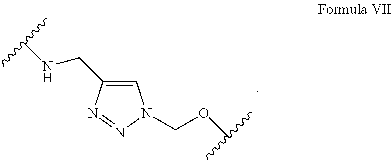 [SMILES: *NCc1cn(CO*)nn1]

16. The kethoxal complex of any one of claims 1 to 15, wherein the agent binds directly or indirectly to a nucleic acid in vivo, ex vivo and/or in vitro.

17. The kethoxal complex of any one of claims 1 to 16, wherein the agent is a therapeutic, diagnostic, or functional agent.

18. The kethoxal complex of claim 17, wherein the therapeutic agent is a small molecule.

19. The kethoxal complex of claim 18, wherein the small molecule binds to a protein or a nucleic acid.

20. The kethoxal complex of any one of claims 1 to 17, wherein the agent is a therapeutic nucleic acid.

21. The kethoxal complex of claim 20, wherein the therapeutic nucleic acid is an inhibitory nucleic acid.

22. The kethoxal complex of claim 20, wherein the inhibitory nucleic acid is an siRNA.

23. The kethoxal complex of claim 1, wherein the kethoxal derivative is N₃-kethoxal.

24. A method for localizing an agent to a nucleic acid comprising contacting a cell or an extracellular nucleic acid with a kethoxal complex of any one of claims 1 to 23.

25. The method of claim 24, wherein the agent is a therapeutic agent.

26. A method for localizing a therapeutic agent in a cell comprising:
(i) contacting a target cell with a kethoxal complex of any one of claims 1 to 16 to form a treated cell; and
(ii) coupling the therapeutic agent to a nucleic acid through a kethoxal derivative-coupled guanine base(s).

27. A kethoxal derivative of Formula VI 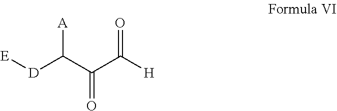
wherein A is H or methyl, D is a linker or a direct bond; and
wherein E is a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene.

28. The kethoxal derivative of claim 27, wherein D is —(CR⁵H)ₙ— where n is 1-10 and R⁵ is H or alkyl such as methyl; —O(CR⁶H)ₘ— where m is 1-10 and R⁶ is H or alkyl such as methyl; —NR⁷— where R⁷ is H or alkyl such as methyl; —NR⁸CO(CR⁹H)ⱼ— where j is 1-10 and R⁸ and R⁹ are independently H or alkyl such as methyl; or —O(CR¹⁰H)ₖR¹¹— where k is 1-10 and R¹⁰ is H or alkyl such as methyl and R¹¹ is alkyl, substituted alkyl, cycloalkyl, substituted cycloalkyl, heteroalkyl, substituted heteroalkyl, aryl, substituted aryl, heteroaryl, or substituted heteroarylaryl.

29. The kethoxal derivative of claim 27, wherein E further comprises a detectable label.

30. The kethoxal derivative of claim 29, wherein the detectable label is a drug, a toxin, a peptide, a polypeptide, an epitope tag, a member of a specific binding pair, a fluorophore, a solid support, a nucleic acid (DNA/RNA), a lipid, or a carbohydrate.

31. The kethoxal derivative of claim 27, wherein E further comprises an affinity group.

32. The kethoxal derivative of claim 31, wherein the affinity group is biotin.
